(19) United States
(12) Patent Application Publication
(10) Pub. No.: US 2022/0257601 A1
(43) Pub. Date: Aug. 18, 2022

(54) INHIBITORS OF PRC1 FOR TREATMENT OF CANCER

(71) Applicants: Board of Regents, The University of Texas System, Austin, TX (US); Memorial Sloan Kettering Cancer Center, New York, NY (US)

(72) Inventors: Filippo Giancotti, Houston, TX (US); Ouathek Ouerfelli, Fort Lee, NJ (US); Wenjing Su, New York, NY (US); Guangli Yang, Syosset, NY (US); Howard Scher, Tenafly, NY (US); Mohammad Marzabadi, Ridgewood, NJ (US)

(73) Assignees: Board of Regents, The University of Texas System, Austin, TX (US); Memorial Sloan Kettering Cancer Center, New York, NY (US)

(21) Appl. No.: 17/622,287

(22) PCT Filed: Jun. 26, 2020
(86) PCT No.: PCT/US2020/039896

§ 371 Date: Dec. 23, 2021

Related U.S. Application Data
(60) Provisional application No. 62/867,760, filed on Jun. 27, 2019.

(51) Int. Cl. A61K 31/519; A61K 31/4406; A61K 31/4035; A61P 35/04; A61K 45/06

(52) U.S. Cl. CPC A61K 31/519; A61K 31/4406; A61K 45/06; A61P 35/04; A61K 31/4035

(57) ABSTRACT

Disclosed herein are compounds and methods for the inhibition of the RNF1 or RNF2 subunit of polycomb repressive complex 1 (PRC1) for the treatment of metastatic cancer, such as metastatic castration-resistant prostate cancer. The inhibitors can be combined with checkpoint inhibitors such as PD-1 inhibitors, PD-L 1 inhibitors, or CTLA-4 inhibitors.

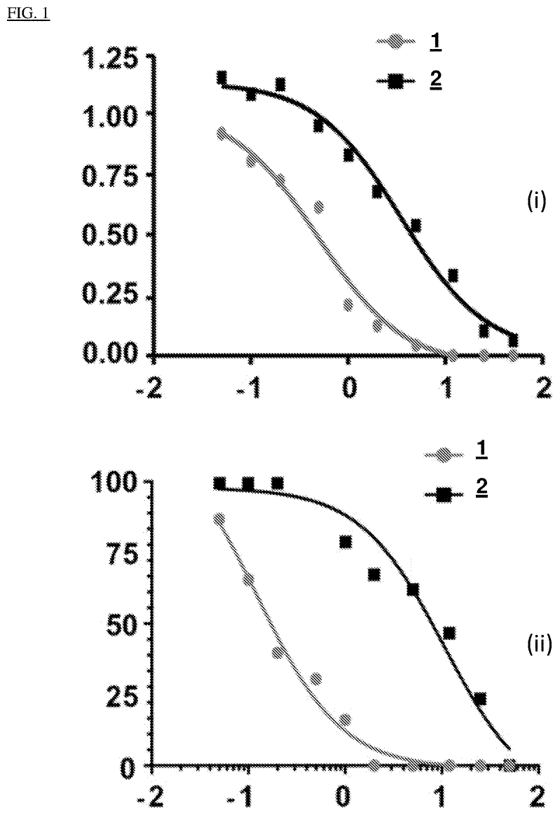

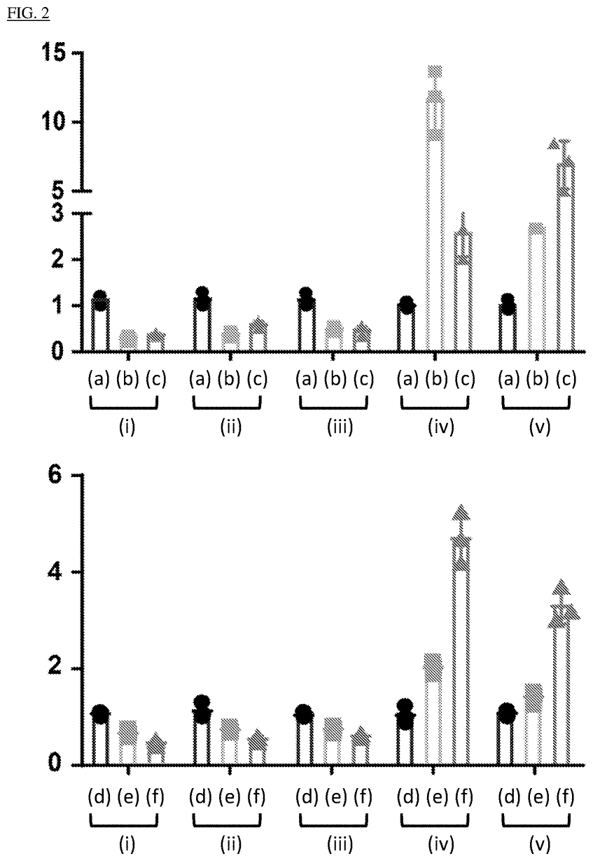

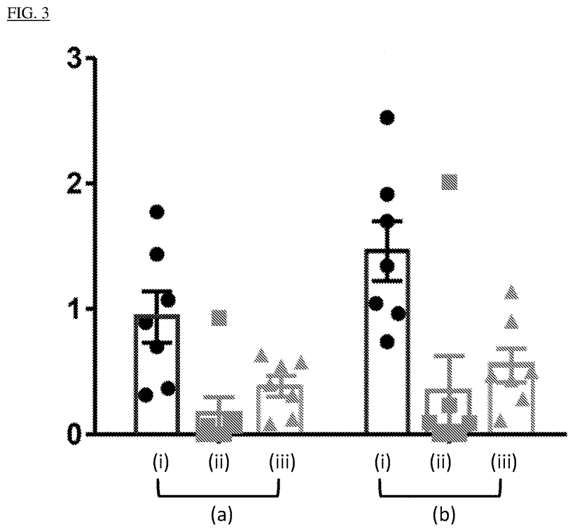

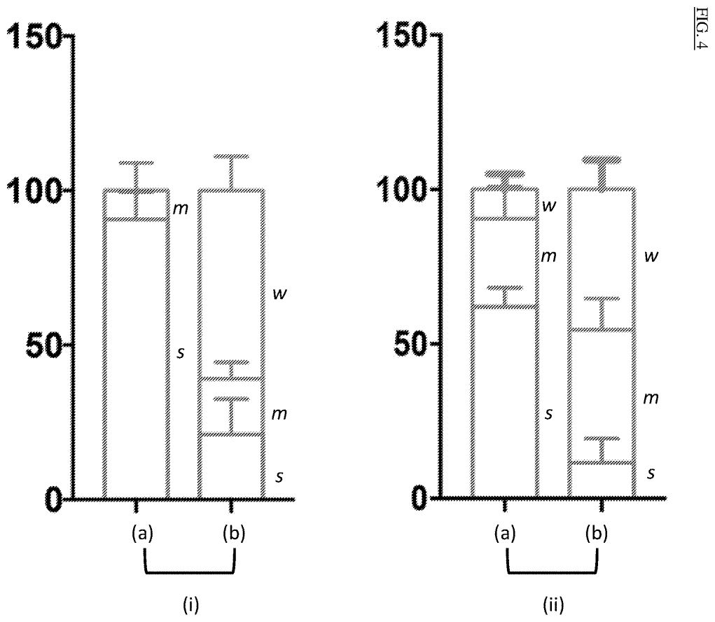

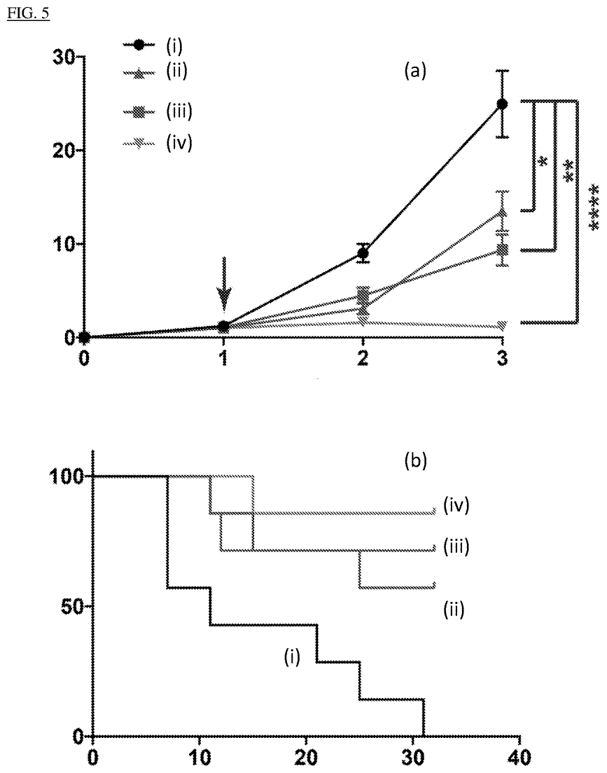

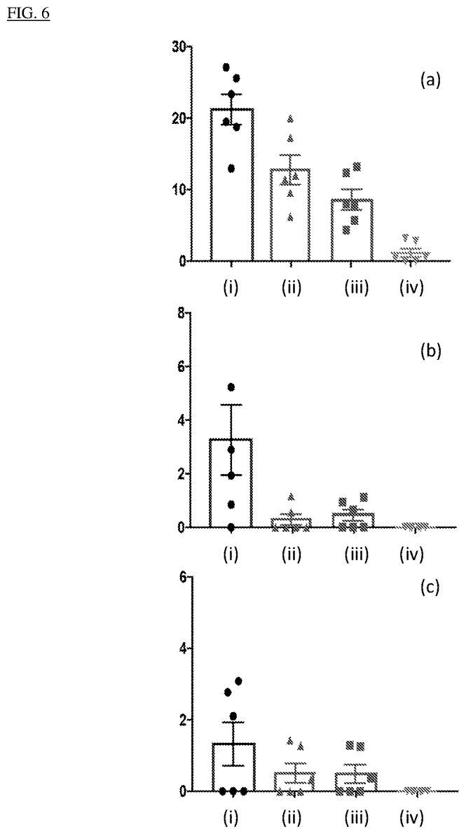

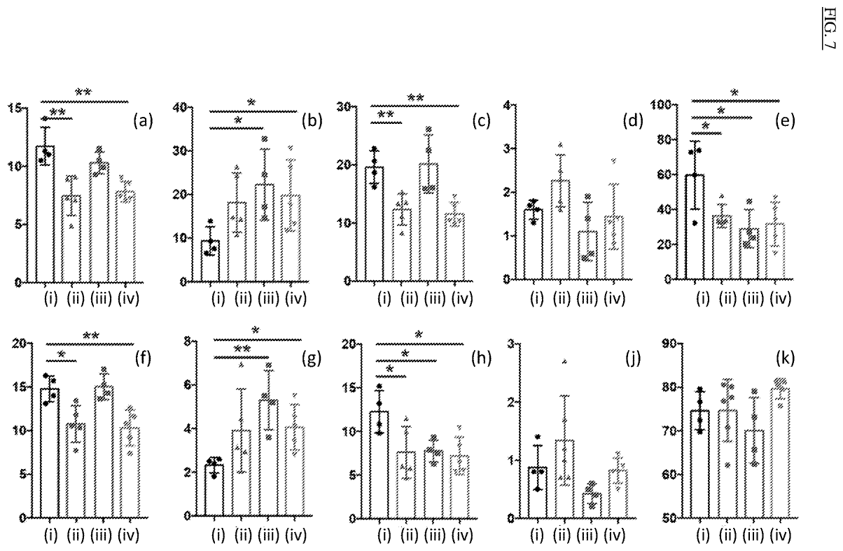

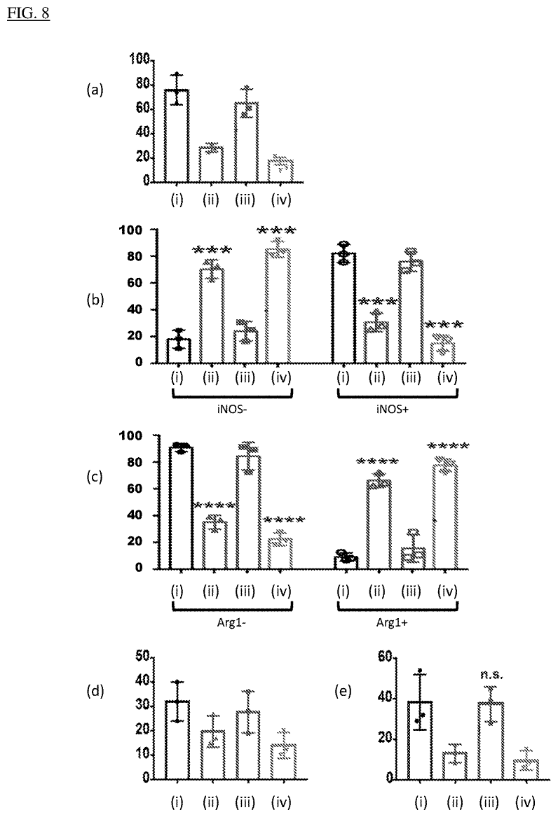

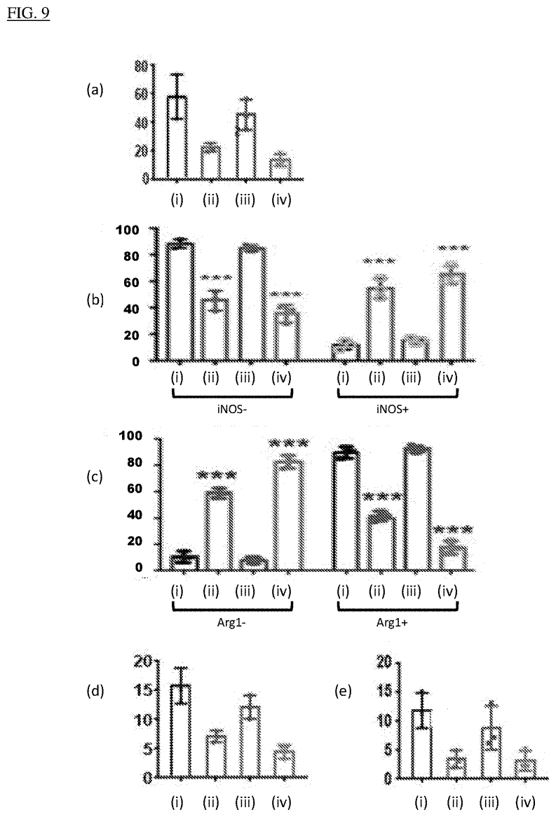

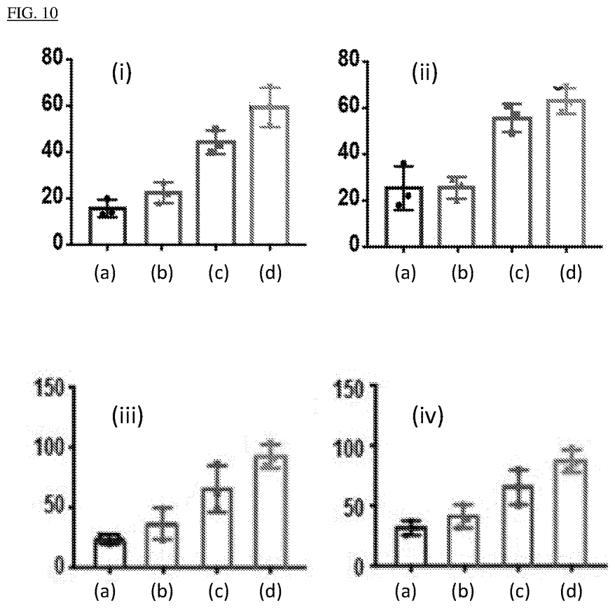

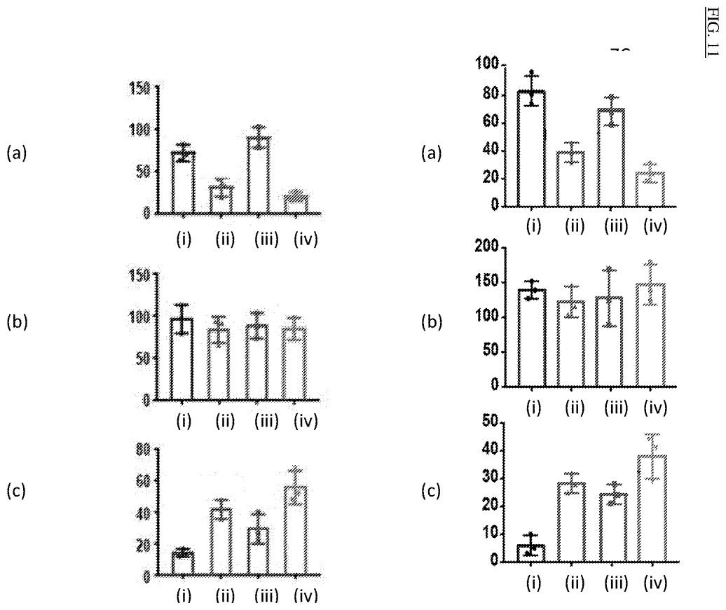

This application claims the benefit of priority of U.S. Provisional Application No. 62/867,760, filed Jun. 27, 2016, the contents of which are incorporated by reference as if written herein in their entirety.

This invention was made with government support under grant number CA197566 awarded by the National Institutes of Health. The government has certain rights in the invention.

Disclosed herein are new compounds and compositions and their application as pharmaceuticals for the treatment of disease. Methods of inhibition of PRC1 activity in a human or animal subject are also provided for the treatment diseases such as cancer.

### BRIEF DESCRIPTION OF THE DISCLOSURE

### Introduction

Cancer cells exploit several mechanisms to evade destruction by the immune system and to resist therapy. However, it is unclear if and to what extent these mechanisms operate also during metastatic colonization of distant organs. Separate lines of inquiry have documented a role for stemness, encompassing both self-renewal and aberrant differentiation, and immune evasion in the outgrowth of metastatic lesions (Giancotti, 2013; Gonzalez et al., 2018). However, it is not known if a common regulatory mechanism orchestrates both functions in support of metastatic colonization.

The mechanisms that enable immune evasion at metastatic sites are poorly understood. Recent studies have attributed the limited efficacy of immunotherapy in CRPC to the presence of an immunosuppressive tumor microenvironment comprising MDSCs and M2-like TAMs (Lu et al., 2017). However, the mechanisms that enable metastatic prostate cancer cells to evade the immune system in target organs are poorly understood.

Chronic inflammation and immunosuppression constitute a significant barrier to the development of effective immunotherapies for metastatic castration-resistant prostate cancer.

TABLE 1
Classification of prostate cancer subtypes.

| Subtypes | Definition |
|---|---|
| NEPC (Beltran et al., | NEPC is defined on the basis of clinical and pathological criteria. |
| 2011; Beltran et al., | Clinically, it manifests as a rapidly progressive and hormone |
| 2014) | refractory disease involving visceral organs, often in the setting of |
| (Neuroendocrine | low or modestly rising serum Prostate Specific Antigen (PSA) |
| Prostate Cancer) | level. |
| | Biopsies performed in this subset may vary, ranging from poorly |
| | differentiated carcinomas to mixed adenocarcinoma-small cell |
| | carcinomas to pure small cell carcinomas. These aggressive |
| | tumors often demonstrate low or absent AR protein expression, |
| | and contain a variable proportion of tumor cells expressing |
| | markers of neuroendocrine differentiation, such as synaptophysin |
| | (SYP) and chromogranin (CHGA). |
| DNPC (Bluemn et | DNPC is defined on the basis of transcriptional profiling as a |
| al., 2017) (Double- | subset of M-CRPC that does not express AR-pathway or |
| Negative Prostate | neuroendocrine genes. It is notable for elevated FGF and MAPK |
| Carcinoma) | pathway activity, which can bypass AR dependence. |
| AVPC (Aparicio et | A subset of prostate cancer that share the clinical, therapy response |
| al., 2016) | and molecular profiles of the small cell prostate carcinomas, a |
| (Aggressive Variant | histological variant of the disease that responds poorly to AR- |
| Prostate Carcinoma) | directed therapies. It is characterized by a molecular signature of |
| | combined tumor suppressor defects (≥2 alterations in Tp53, RB1 |
| | and/or PTEN by immunohistochemistry or genomic analyses). |

In prostate cancer, resistance to hormone deprivation therapy is intimately linked to the development of metastasis. Potent AR inhibitors, such as enzalutamide and abiraterone, can induce durable responses in a fraction of metastatic castration-resistant prostate cancer (M-CRPC) patients. However, the remainder exhibits a transient and often partial response or are completely insensitive to the therapy (Attard et al., 2016). Experiments in model systems suggest that both de novo and acquired resistance can arise from inactivation of TP53 and exposure to abiraterone or simultaneous inactivation of TP53 and RBI1, which can reprogram prostate adenocarcinomas to AR-negative neuroendocrine prostate cancer (NEPC) (Mu et al., 2017). Moreover, experiments with LNCaP-AR cells have specifically implicated the Polycomb Repressive Complex 2 (PRC2, comprising EED, EZH2 and SUZ12) in transdifferentiation to NEPC and resistance to enzalutamide (Ku et al., 2017). However, as the use of abiraterone and enzalutamide in the clinic has become widespread, the incidence of AR pathway-negative M-CRPC devoid of neuroendocrine traits (Double Negative Prostate Cancer, DNPC; see Table S1 for definitions) has risen substantially, highlighting the need to understand the origin and therapeutic vulnerabilities of these cancers (Bluemn et al., 2017).

PRC1 performs complex roles in gene regulation. In addition to the canonical complex (“cPRC1”), biochemical and functional analysis has defined several ncPRC1 complexes (“ncPRC1”), including the cancer-relevant KDM2B-PRC1 complex (ncPRC1.1), which has been found at the promoters of both repressed and actively transcribed genes (Banito et al., 2018; Van den Boom et al., 2016).

Both cPRC1 and ncPRC1 consist of several subunits, each encoded by multiple paralogs, and share the ability to promote monoubiquitination of histone H2A through their common catalytic subunit RNF2. Often acting in tandem to silence target genes, PRC1 and PRC2 promote de-differentiation and stemness during development and in cancer (Schuettengruber et al., 2017). Mouse genetic studies have specifically implicated the cPRC1 component BMI1 in prostate development and malignant transformation (Lukacs et al., 2010). However, the role of both cPRC1 and ncPRC1 activity in prostate cancer progression and metastasis has remained poorly understood.

On the basis of the specific evidence implicating TAMs in bone metastasis, various approaches to target macrophages are in development (Camacho and Pienta, 2014). Although antibodies blocking CCL2 should provide the added benefit of inhibiting the self-renewal capacity of cancer stem cells and the recruitment of TAMs, this approach has not proven to be effective in prostate cancer due to rebound production of CCL2 upon cessation of therapy (Pienta et al., 2013). Importantly, newly produced CCL2 releases inflammatory monocytes from the bone marrow and promotes angiogenesis and metastasis in mouse models of breast cancer, suggesting that anti-CCL2 monotherapy may paradoxically have harmful consequences (Bonapace et al., 2014).

### SUMMARY

In this study, genetically engineered transplantation models of DNPC have been leveraged to show that PRC1 not only controls self-renewal and metastasis initiation but also governs the recruitment of myeloid-derived suppressor cells (“MDSCs”), tumor-associated macrophages (“TAMs”) and regulatory T cells (“Tregs”), thus creating a profoundly immunosuppressive and pro-angiogenic microenvironment in the bone and other metastatic sites. Consistently, pharmacological inhibition of PRC1 reversed these processes and cooperated with double checkpoint immunotherapy (“DCIT”) to suppress multi-organ metastasis. These results reveal a link between epigenetic regulation of stemness and molding of an immunosuppressive microenvironment and identify PRC1 as a therapeutic target in M-CRPC.

It is disclosed herein that PRC1 drives colonization of the bones and visceral organs in Double-Negative Prostate Cancer (DNPC; AR-null NE-null). In vivo genetic screening identifies CCL2 as the top pro-metastatic gene induced by PRC1. Mechanistic studies show that CCL2 governs self-renewal and induces the recruitment of M2-like TAMs and Tregs, thus coordinating metastasis initiation with immunosuppression and neoangiogenesis. These results reveal a link between epigenetic regulation of cancer stem cells and molding of the tumor microenvironment, and more specifically reveal that PRC1 coordinates stemness with immune evasion and neoangiogenesis.

Herein is provided evidence that PRC1 promotes metastatic spread to the bone and visceral organs in DNPC. Finally, it is demonstrated that pharmacological inhibition of PRC1 with a novel catalytic inhibitor of RNF2, in combination with checkpoint immunotherapy, suppresses multi-organ site metastasis in preclinical genetically-engineered transplantation models that mimic human DNPC, pointing to the potential clinical utility of targeting PRC1 in M-CRPC.

### BRIEF DESCRIPTION OF THE SEVERAL VIEW OF THE DRAWINGS

FIG. Error! Bookmark not defined. shows the treatment of PC3 cells by Compound 1 and Compound 2. Horizontal axis=log₁₀ (conc, μM). (i) Inhibition of H2Aub normalized to control groups and determination of IC₅₀ value: IC₅₀(1)=470 nm; IC₅₀(2)=3.52 μM. (ii) Inhibition of sphere formation ability normalized to control groups and determination of IC₅₀ value: IC₅₀(Compound 1)=130 nm; IC₅₀(Compound 2)=1.054 μM.

FIG. Error! Bookmark not defined. shows quantitative RT-PCR analysis of mRNA levels of RNF2 target genes upon RNF2 knockdown or treatment with Compound 1 in (upper) PC3 or (lower) RM1 cells. Vertical axis=relative mRNA levels. PRC1-induced: (i) CCL2, (ii) CXCL1, (iii) LGR5; PRC1-repressed: (iv) NTS, (v) ATF3. (a) and (d): control; (b): RNF2 knockdown; (e): 0.5 μM Compound 1; (d) and (f): 1.0 μM Compound 1.

FIG. Error! Bookmark not defined. shows normalized photon flux (1×10⁹) of male nude mice injected intracardially with 2.5×10⁵ PC3 cells at (a) 4 weeks; (b) 5 weeks; (i) vehicle; (ii) 2×/week treatment with Compound 1 from day 7; (iii) 2×/week treatment with Compound 1 from day 21; bars: SEM; P<0.05.

FIG. Error! Bookmark not defined. shows IHC staining of bone tissue from the mice of FIG. Error! Bookmark not defined, using (i) anti-CCL2 and (ii) anti-UbH2A antibodies. (a) vehicle; (b) Compound 1; staining intensities classified as: w=weak or absent, m=moderate, or s=strong.

FIG. Error! Bookmark not defined. shows (a) photon flux (horizontal axis=weeks; treatment initiated at week 1) and (b) survival curve (horizontal axis=days) for male FBV mice injected intracardially with 1×10⁵ luciferase labelled Pten^{PC-/-}Smad4^{PC-/-} cells. (i) vehicle; (ii) Compound 1; (iii) CTLA4+PD1; (iv) 1+CTLA4+PD1.

FIG. Error! Bookmark not defined. shows quantification of luciferase counts at day 21 post injection for (a) bone, (b), liver, and (c) brain for the mice from FIG. Error! Bookmark not defined. (i) vehicle; (ii) Compound 1; (iii) CTLA4+PD1; (iv) Compound 1+CTLA4+PD1. Bars=SEM.

FIG. Error! Bookmark not defined. shows FACS analysis of immune cell population for the mice from FIG. Error! Bookmark not defined. (i) vehicle; (ii) Compound 1; (iii) CTLA4+PD1; (iv) Compound 1+CTLA4+PD1. Blood: (a) macrophage F4/80+; (b) T cell CD3+; (c) M-MDSC CD11b/Ly6C^{high}/LyCg^{low}; (d) NK cell NK1.1+; (e) Neutrophil CD11b/Gr-1+. Bone marrow: (f) macrophage F4/80+; (g) T cell CD3+; (h) M-MDSC CD11b/Ly6C^{high}/LyCg^{low}; (j) NK cell NK1.1+; (k) Neutrophil CD11b/Gr-1+.

FIG. Error! Bookmark not defined. shows quantification of positive cells from mice injected with Pten^{pc-/-}Smad4^{pc-/-} cells. (a) CD68+, y-axis=no./field; (b) iNOS− (left)/iNOS+ (right), y-axis=% of CD68+; (c) Arg1− (left)/Arg1+ (right), y-axis=% of CD68+; (d) Foxp3+, y-axis=no./field; (e)=B220+, y-axis=no./field. Bars=SD; **** P<0.0001

FIG. Error! Bookmark not defined. shows quantification of positive cells from mice injected with RM1 cells. (a) CD68+, y-axis=no./field; (b) iNOS− (left)/iNOS+ (right), y-axis=% of CD68+; (c) Arg1− (left)/Arg1+(right), y-axis=% of CD68+; (d) Foxp3+, y-axis=no./field; (e)=B220+, y-axis=no./field. Bars=SD; **** P<0.0001

FIG. Error! Bookmark not defined. shows quantification of positive cells from bone tissues (bars=SD; **** P<0.0001) from mice injected with (i and ii) Pten^{pc-/-}Smad4^{pc-/-} and (iii and iv) RM1 cells. Samples were collected after 1 week treatment and subjected to IHC or IF staining: (i and iii) CD4/H; (iii and iv) CD8/H; (a) vehicle; (b) Compound 1; (c) CTLA4+PD1; (d) Compound 1+CTLA4+PD1; y-axis=no/field.

FIG. Error! Bookmark not defined. shows quantification of positive cells from bone tissues (bars=SD; **** P<0.0001) from mice injected with Pten^{pc-/-}Smad4^{pc-/-} cells or RM1 cells. Samples were collected after 1 week treatment and subjected to IHC or IF staining. (i) vehicle; (ii) Compound 1; (iii) CTLA4+PD1; (iv) Compound 1+CTLA4+PD1; (a) CD31/H; (b) Ki67/H; (c) CC3/H. y-axis=no/field. The graphs on the right are from data in Pten^{pc-/-}Smad4^{pc-/-} cells; the graphs on the left are from data in RM1 cells.

### DETAILED DESCRIPTION

Provided herein is Embodiment 1, a compound of structural Formula (I)

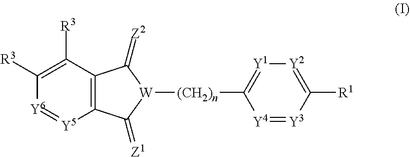

or a salt or tautomer thereof, wherein:
n is chosen from 2, 3, and 4;
W is chosen from CH and N;
Y¹, Y², Y³, and Y⁴ are independently chosen from C(R²) and N;
Y⁵, and Y⁶ are independently chosen from C(R³) and N;
Z¹ and Z² are independently chosen from ═O, ═S, —H/—OH, and —H/—H;
R¹ is chosen from amino, hydroxy, cyano, halo, alkyl, cycloalkyl, heterocycloalkyl, aryl, heteroaryl, alkoxy, cycloalkoxy, heterocycloalkoxy, aryloxy, and heteroaryloxy, any of which is optionally substituted with one or more R⁴ groups;
each R² is independently chosen from H, halo, amino, cyano, and hydroxy;
each R³ is independently chosen from H, halo, amino, cyano, and hydroxy; and
each R⁴ is independently chosen from alkyl, alkoxy, alkoxyalkyl, alkylcarbonyl, alkylsulfonyl, amino, aminocarbonyl, cyano, carboxy, halo, haloalkoxy, haloalkyl, hydroxy, hydroxyalkyl, and oxo.

Certain compounds disclosed herein may possess useful PRC1 inhibiting activity, and may be used in the treatment or prophylaxis of a disease or condition in which PRC1 plays an active role. Thus, in broad aspect, certain embodiments also provide pharmaceutical compositions comprising one or more compounds disclosed herein together with a pharmaceutically acceptable carrier, as well as methods of making and using the compounds and compositions. Certain embodiments provide methods for inhibiting PRC1. Other embodiments provide methods for treating a PRC1-mediated disorder in a patient in need of such treatment, comprising administering to said patient a therapeutically effective amount of a compound or composition according to the present invention. Also provided is the use of certain compounds disclosed herein for use in the manufacture of a medicament for the treatment of a disease or condition ameliorated by the inhibition of PRC1.

Also provided are the following embodiments:

Embodiment 2: the compound of Embodiment 1, wherein R¹ is chosen from amino, hydroxy, cyano, halo, alkyl, cycloalkyl, heterocycloalkyl, aryl, and heteroaryl, any of which is optionally substituted with 1, 2, or 3 R⁴ groups.

Embodiment 3: the compound of Embodiment 2, wherein R¹ is chosen from amino, hydroxy, cyano, halo, alkyl, cycloalkyl, and heterocycloalkyl, any of which is optionally substituted with 1, 2, or 3 R⁴ groups.

Embodiment 4: the compound of Embodiment 3, wherein R¹ is chosen from amino, alkyl, cycloalkyl, and heterocycloalkyl, any of which is optionally substituted with 1, 2, or 3 R⁴ groups.

Embodiment 5: the compound of any one of Embodiments 1-4, wherein R¹ is optionally substituted with 1 or 2 R⁴ groups.

Embodiment 6: the compound of Embodiment 5, wherein R¹ is optionally substituted with 1 R⁴ group.

Embodiment 7: the compound of Embodiment 6, wherein R¹ is substituted with 1 R⁴ group.

Embodiment 8: the compound of any one of Embodiments 1-7, wherein each R⁴ is independently chosen from alkyl, alkylcarbonyl, alkylsulfonyl, amino, aminocarbonyl, cyano, carboxy, halo, haloalkyl, hydroxy, and oxo.

Embodiment 9: the compound of Embodiment 8, wherein each R⁴ is independently chosen from alkyl, amino, cyano, halo, haloalkyl, hydroxy, and oxo.

Embodiment 10: the compound of Embodiment 9, wherein each R⁴ is independently chosen from alkyl, NH₂, cyano, halo, haloalkyl, and hydroxy.

Embodiment 11: the compound of Embodiment 10, wherein each R⁴ is independently chosen from NH₂, cyano, halo, and hydroxy.

Embodiment 12: the compound of Embodiment 6, wherein R¹ is not substituted with an R⁴ group.

Embodiment 13: the compound of any one of Embodiments 1-12, wherein Y¹ is N.

Embodiment 14: the compound of any one of Embodiments 1-12, wherein Y¹ is C(R²).

Embodiment 15: the compound of any one of Embodiments 1-14, wherein Y² is N.

Embodiment 16: the compound of any one of Embodiments 1-14, wherein Y² is C(R²).

Embodiment 17: the compound of any one of Embodiments 1-16, wherein Y³ is N.

Embodiment 18: the compound of any one of Embodiments 1-16, wherein Y³ is C(R²).

Embodiment 19: the compound of any one of Embodiments 1-18, wherein Y⁴ is N.

Embodiment 20: the compound of any one of Embodiments 1-18, wherein Y⁴ is C(R²).

Also provided herein is Embodiment 21, a compound of structural Formula (II)

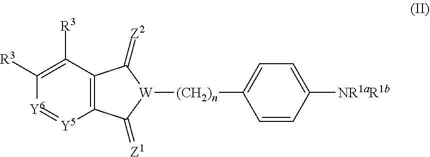

or a salt or tautomer thereof, wherein:
n is chosen from 2, 3, and 4;
W is chosen from CH and N;
Y⁵ and Y⁶ are independently chosen from C(R³) and N;
Z¹ and Z² are independently chosen from ═O, ═S, —H/—OH, and —H/—H;
R^{1a} and R^{1b} are independently chosen from hydrogen, alkyl, acyl, heteroalkyl, aryl, cycloalkyl, heteroaryl, and heterocycloalkyl, any of which is optionally substituted with one or more R⁴ groups,
or R^{1a} and R^{1b}, together with the intervening nitrogen, combine to form a 3-7 membered heterocycloalkyl, which is optionally substituted with one or more R⁴ groups;
each R³ is independently chosen from H, halo, amino, cyano, and hydroxy; and
each R⁴ is independently chosen from alkyl, alkoxy, alkoxyalkyl, alkylcarbonyl, alkylsulfonyl, amino, aminocarbonyl, cyano, carboxy, halo, haloalkoxy, haloalkyl, hydroxy, hydroxyalkyl, and oxo.

Embodiment 22: the compound of Embodiment 21, wherein R^{1a} and R^{1b} are independently chosen from hydrogen, alkyl, acyl, heteroalkyl, aryl, cycloalkyl, heteroaryl, and heterocycloalkyl, any of which is optionally substituted with 1, 2, or 3 R⁴ groups.

Embodiment 23: the compound of Embodiment 22, wherein R^{1a} and R^{1b} are independently chosen from hydrogen, alkyl, and acyl, any of which is optionally substituted with 1, 2, or 3 R⁴ groups.

Embodiment 24: the compound of Embodiment 23, wherein R^{1a} and R^{1b} are independently chosen from alkyl and acyl, either of which is optionally substituted with 1, 2, or 3 R⁴ groups.

Embodiment 25: the compound of any one of Embodiments 22-24, wherein each of R^{1a} and R^{1b} is optionally substituted with 1 or 2 R⁴ groups.

Embodiment 26: the compound of Embodiment 25, wherein each of R^{1a} and R^{1b} is optionally substituted with 1 R⁴ group.

Embodiment 27: the compound of Embodiment 21, wherein R^{1a} and R^{1b}, together with the intervening nitrogen, combine to form a 3-7 membered heterocycloalkyl, which is optionally substituted with 1, 2, or 3 R⁴ groups.

Embodiment 28: the compound of Embodiment 27, wherein R^{1a} and R^{1b}, together with the intervening nitrogen, combine to form a 4-6 membered heterocycloalkyl, which is optionally substituted with 1, 2, or 3 R⁴ groups.

Embodiment 29: the compound of either of Embodiments 27 and 28, wherein the heterocycloalkyl formed by R^{1a} and R^{1b}, together with the intervening nitrogen, is optionally substituted with 1 or 2 R⁴ groups.

Embodiment 30: the compound of Embodiment 29, wherein the heterocycloalkyl formed by R^{1a} and R^{1b}, together with the intervening nitrogen, is optionally substituted with 1 R⁴ group.

Embodiment 31: the compound of any one of Embodiments 21-30, wherein Y⁵ is N.

Embodiment 32: the compound of any one of Embodiments 21-30, wherein Y⁵ is C(R²).

Embodiment 33: the compound of any one of Embodiments 21-32, wherein Y⁶ is N.

Embodiment 34: the compound of any one of Embodiments 21-32, wherein Y⁶ is C(R²).

Embodiment 35: the compound of any one of Embodiments 1-34, wherein each R² is independently chosen from H, halo, and hydroxy.

Embodiment 36: the compound of Embodiment 35, wherein each R² is independently chosen from H and halo.

Embodiment 37: the compound of Embodiment 36, wherein each R² is independently chosen from H, F, Cl, and Br.

Embodiment 38: the compound of Embodiment 37, wherein each R² is independently chosen from H, F, and Cl.

Embodiment 39: the compound of Embodiment 38, wherein each R² is independently chosen from H and F.

Embodiment 40: the compound of any one of Embodiments 1-39, wherein at least one R² is chosen from halo, NH₂, cyano, and hydroxy.

Embodiment 41: the compound of Embodiment 40, wherein at least one R² is chosen from halo and hydroxy.

Embodiment 42: the compound of Embodiment 40, wherein at least one R² is chosen from F, Cl, and Br.

Embodiment 43: the compound of any one of Embodiments 1-42, wherein each R³ is independently chosen from H, halo, and hydroxy.

Embodiment 44: the compound of Embodiment 43, wherein each R³ is independently chosen from H and halo.

Embodiment 45: the compound of Embodiment 44, wherein each R³ is independently chosen from H, F, Cl, and Br.

Embodiment 46: the compound of Embodiment 45, wherein each R³ is independently chosen from H, F, and Cl.

Embodiment 47: the compound of Embodiment 46, wherein each R³ is independently chosen from H and F.

Embodiment 48: the compound of any one of Embodiments 1-47, wherein at least one R² is chosen from halo, NH₂, cyano, and hydroxy.

Embodiment 49: the compound of Embodiment 48, wherein at least one R³ is chosen from halo and hydroxy.

Embodiment 50: the compound of Embodiment 48, wherein at least one R³ is chosen from F, Cl, and Br.

Embodiment 51: the compound of any one of Embodiments 1-50, wherein W is N.

Embodiment 52: the compound of any one of Embodiments 1-50, wherein W is CH.

Embodiment 53: the compound of either one of Embodiments 51 and 52, wherein Z¹ and Z² are independently chosen from ═O and ═S.

Embodiment 54: the compound of Embodiment 53, wherein Z¹ and Z² are ═O.

Embodiment 55: the compound of Embodiment 53, wherein Z¹ and Z² are ═S.

Embodiment 56: the compound of either one of Embodiments 51 and 52, wherein at least one of Z¹ and Z² is-H/—H.

Embodiment 57: the compound of Embodiment 56, wherein exactly one of Z¹ and Z² is ═O.

Embodiment 58: the compound of Embodiment 56, wherein exactly one of Z¹ and Z² is ═S.

Embodiment 59: the compound of Embodiment 56, wherein Z¹ and Z² are-H/—H.

Embodiment 60: the compound of Embodiment 51, wherein at least one of Z¹ and Z² is-H/—OH.

Embodiment 61: the compound of Embodiment 60, wherein exactly one of Z¹ and Z² is ═O.

Embodiment 62: the compound of Embodiment 60, wherein exactly one of Z¹ and Z² is ═S.

Embodiment 63: the compound of Embodiment 60, wherein Z¹ and Z² are-H/—OH.

Embodiment 64: the compound of any one of Embodiments 1-63, wherein n is chosen from 2 and 3.

Embodiment 65: the compound of Embodiment 64, wherein n is 2.

Embodiment 66: the compound of any one of Embodiments 1-65, wherein the compound is a PRC inhibitor.

Embodiment 67: the compound of Embodiment 66, wherein the compound exhibits an IC₅₀ for PRC1 of <20 μM.

Embodiment 68: the compound of Embodiment 67, wherein the compound exhibits an IC₅₀ for PRC1 of <10 μM.

Embodiment 69: the compound of Embodiment 68, wherein the compound exhibits an IC₅₀ for PRC1 of <5 μM.

Embodiment 70: the compound of Embodiment 69, wherein the compound exhibits an IC₅₀ for PRC1 of <1 μM.

Embodiment 71: the compound of any one of Embodiments 1-65, wherein the compound is a PRC catalytic inhibitor.

Embodiment 72: the compound of Embodiment 71, wherein the compound exhibits an IC₅₀ for either one of RNF1 and RNF2 of <100 μM.

Embodiment 73: the compound of Embodiment 72, wherein the compound exhibits an IC₅₀ for either one of RNF1 and RNF2 of <50 μM.

Embodiment 74: the compound of Embodiment 73, wherein the compound exhibits an IC₅₀ for either one of RNF1 and RNF2 of <20 μM.

Embodiment 75: the compound of Embodiment 74, wherein the compound exhibits an IC₅₀ for either one of RNF1 and RNF2 of <10 μM.

Embodiment 76: the compound of Embodiment 75, wherein the compound exhibits an IC₅₀ for either one of RNF1 and RNF2 of <5 μM.

Embodiment 77: the compound of Embodiment 76, wherein the compound exhibits an IC₅₀ for either one of RNF1 and RNF2 of <1 μM.

Embodiment 78: the compound of Embodiment 1, wherein the compound is 2-(4-aminophenethyl)isoindoline-1,3-dione.

Embodiment 79: A compound of chosen from 2-(4-aminophenethyl)isoindoline-1,3-dione, 2-(pyridin-3-ylmethylene)-1H-indene-1,3(2H)-dione, and N-(2,6-dibromo-4-methoxyphenyl)-4-(2-methylimidazo[1,2-a]pyrimidin-3-yl)thiazol-2-amine.

Also provided herein is Embodiment M-1: method for the treatment of cancer in a subject in need thereof, the method comprising the administration of a therapeutically effective amount of a compound as disclosed herein, or a salt or tautomer thereof, to a patient in need thereof. For example, the compound may be any one of those disclosed in Embodiments 1-79.

Also provided are the following embodiments:

Embodiment M-2: the method of Embodiment M-1, wherein the cancer is prostate cancer.

Embodiment M-3: the method of Embodiment M-2, wherein the prostate cancer is metastatic castration-resistant prostate cancer.

Embodiment M-4: the method of Embodiment M-2, wherein the prostate cancer is androgen receptor pathway active prostate cancer.

Embodiment M-5: the method of Embodiment M-2, wherein, the prostate cancer is neuroendocrine prostate cancer.

Embodiment M-6: the method of Embodiment M-2, wherein, the prostate cancer is double negative prostate cancer.

Also provided herein is Embodiment M-7: a method for reducing the degree of metastasis of metastatic castration-resistant prostate cancer in a subject in need thereof, the method comprising the administration of a therapeutically effective amount of a compound as disclosed herein, or a salt or tautomer thereof, to a patient in need thereof.

Also provided herein is Embodiment M-8: a method for reducing the plasma level of one or more cytokines in a subject in need thereof, the method comprising the administration of a therapeutically effective dose of a therapeutically effective amount of a compound as disclosed herein, or a salt or tautomer thereof, to a patient in need thereof.

Also provided herein is Embodiment M-9: a method for reducing angiogenesis in a subject in need thereof, the method comprising the administration of a therapeutically effective amount of a compound as disclosed herein, or a salt or tautomer thereof, to a patient in need thereof.

Also provided herein is Embodiment M-10: a method for reducing immunosuppression in a subject in need thereof, the method comprising the administration of a therapeutically effective amount of a compound as disclosed herein, or a salt or tautomer thereof, to a patient in need thereof.

Also provided is Embodiment M-11: a method for reducing the expression of a chemokine in a subject in need thereof, the method comprising the administration of a therapeutically effective amount of a compound as disclosed herein, or a salt or tautomer thereof, to a patient in need thereof. In certain embodiments, the chemokine is a CC chemokine. In certain embodiments, the CC chemokine is CCL2.

Also provided herein is Embodiment M-12: a method for inhibiting and/or reducing cancer stem cells in a subject in need thereof, the method comprising the administration of a therapeutically effective amount of a compound as disclosed herein, or a salt or tautomer thereof, to a patient in need thereof.

Also provided herein is Embodiment M-13: a method for reducing chemoresistance in a subject in need thereof, the method comprising the administration of a therapeutically effective amount of a compound as disclosed herein, or a salt or tautomer thereof, to a patient in need thereof.

Also provided are the following embodiments:

Embodiment M-14: The method of any one of Embodiments M-1-M-13, wherein the compound as disclosed herein is a PRC inhibitor.

Embodiment M-15: The method of Embodiments M-14, wherein the compound as disclosed herein exhibits an IC₅₀ for PRC1 of <20 μM.

Embodiment M-16: The method of Embodiments M-15, wherein the compound as disclosed herein exhibits an IC₅₀ for PRC1 of <10 μM.

Embodiment M-17: The method of Embodiments M-16, wherein the compound as disclosed herein exhibits an IC₅₀ for PRC1 of <5 μM.

Embodiment M-18: The method of Embodiments M-17, wherein the compound as disclosed herein exhibits an IC₅₀ for PRC1 of <1 μM.

Embodiment M-19: The method of any one of Embodiments M-1-M-13, wherein the compound as disclosed herein is a PRC catalytic inhibitor.

Embodiment M-20: The method of Embodiments M-19, wherein the compound as disclosed herein inhibits either of RNF1 or RNF2 with an IC₅₀ of <50 μM.

Embodiment M-21: The method of Embodiments M-20, wherein the compound as disclosed herein inhibits either of RNF1 or RNF2 with an IC₅₀ of <20 μM.

Embodiment M-22: The method of Embodiments M-21, wherein the compound as disclosed herein exhibits an IC₅₀ for either one of RNF1 and RNF2 of <10 μM.

Embodiment M-23: The method of Embodiments M-22, wherein the compound as disclosed herein exhibits an IC₅₀ for either one of RNF1 and RNF2 of <5 μM.

Embodiment M-24: The method of Embodiments M-23, wherein the compound as disclosed herein exhibits an IC₅₀ for either one of RNF1 and RNF2 of <1 μM.

For clarity, also provided are embodiments wherein the compound recited in any of Embodiments M1-M24 is a compound as recited in any of Embodiments 1-79.

In certain embodiments of each of the above methods, the method further comprises the coadministration of one or more checkpoint inhibitors. In certain embodiments, the one or more checkpoint inhibitors comprises one or more CTLA-4 inhibitors. In certain embodiments, the one or more checkpoint inhibitors comprises one or more CTLA-4 inhibitors. In certain embodiments, the one or more checkpoint inhibitors comprises one or more PD-1 inhibitors. In certain embodiments, the one or more checkpoint inhibitors comprises one or more PD-L1 inhibitors. In certain embodiments, the one or more checkpoint inhibitors comprises a CTLA4 inhibitor and a PD-1 inhibitor. In certain further embodiments, the checkpoint inhibitor is chosen from nivolumab, pembrolizumab, and ipilimumab.

Specifically, also provided herein are Embodiments C-1-C-24, comprising the methods recited in Embodiments M-1-M-24 and further comprising the coadministration of one or more checkpoint inhibitors.

Also provided are the following embodiments:

Embodiment C-25: the method of any of Embodiments C-1-C-24, wherein the one or more checkpoint inhibitors comprises one or more CTLA-4 inhibitors.

Embodiment C-26: the method of C-25, wherein the one or more checkpoint inhibitors comprises one or more CTLA-4 inhibitors.

Embodiment C-27: the method of C-25, wherein the one or more checkpoint inhibitors comprises one or more PD-1 inhibitors.

Embodiment C-28: the method of C-25, wherein the one or more checkpoint inhibitors comprises one or more PD-L1 inhibitors.

Embodiment C-29: the method of C-25, wherein the one or more checkpoint inhibitors comprises a CTLA4 inhibitor and a PD-1 inhibitor.

Embodiment C-30: the method of C-25, wherein the checkpoint inhibitor is chosen from nivolumab, pembrolizumab, and ipilimumab.

For clarity, also provided are embodiments corresponding to any of the above embodiments, wherein is provided the use of a compound of any Embodiments 1-79 in the method as recited in any of Embodiments M1-M24 and C₁-C₃₀; or wherein is provided a compound of any Embodiments 1-79 in for use in the manufacture of a medicament for the method as recited in any of Embodiments M1-M24 and C₁-C₃₀; or wherein is provided a pharmaceutical composition comprising a compound of any Embodiments 1-79, optionally for use in the method as recited in any of Embodiments M1-M24 and C₁-C₃₀.

### Abbreviations

AR=androgen receptor; ARPC=androgen receptor pathway active prostate cancer; bFGF=basic fibroblast growth factor; BMI1=B-lymphoma Moloney murine leukemia virus insertion region 1; CCL2=C—C motif chemokine ligand 2; cPRC1=canonical PRC1; ncPRC1=non canonical PRC1; DCIT=double checkpoint immunotherapy; DNPC=double negative prostate cancer; EGF=epidermal growth factor; EMT=epithelial-mesenchymal transition; FACS=fluorescence-activated cell sorting; FBS=fetal bovine serum; FDR=false discovery rate; GO=Gene Ontology; GSEA=gene set enrichment analysis; HBSS=Hank's Balanced Salt Solution; IKK=IκB kinase; KEGG=Kyoto Encyclopedia of Genes and Genomes; M-CPRC=metastatic castration-resistant prostate cancer; MDSC=myeloid-derived suppressor cell; MTT=3-(4,5-dimethylthiazol-2-yl)-2,5-diphenyl tetrazolium bromide; NEPC=neuroendocrine prostate cancer; PBS=phosphate buffered saline; PRC=polycomb repressive complex; PrEGM=prostate epithelial cell growth medium; RIPA=radioimmunoprecipitation assay; RNF1=ring finger protein 1; RNF2=ring finger protein 2; TAM=tumor-associated macrophage; TCGA=The Cancer Genome Atlas Program; Treg=regulatory T cell; UBCH5c=ubiquitin-conjugating enzyme H5c.

### Definitions

As used herein, the terms below have the meanings indicated.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 8 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(O)N(RR′) group with R and R′ as defined herein or as defined by the specifically enumerated “R” groups designated. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently chosen from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, naphthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronaphthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or combinations thereof, fully saturated or containing from 1 to 3 degrees of unsaturation, consisting of the stated number of carbon atoms and from one to three heteroatoms chosen from N, O, and S, and wherein the N and S atoms may optionally be oxidized and the N heteroatom may optionally be quaternized. The heteroatom(s) may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom chosen from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzindolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated (but nonaromatic) monocyclic, bicyclic, or tricyclic heterocyclic group containing at least one heteroatom as a ring member, wherein each said heteroatom may be independently chosen from nitrogen, oxygen, and sulfur. In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently chosen from carbon, nitrogen, oxygen and sulfur.

The term “lower,” as used herein, alone or in a combination, where not otherwise specifically defined, means containing from 1 to and including 6 carbon atoms (i.e., C₁-C₆ alkyl).

The term “lower aryl,” as used herein, alone or in combination, means phenyl or naphthyl, either of which may be optionally substituted as provided.

The term “lower heteroaryl,” as used herein, alone or in combination, means either 1) monocyclic heteroaryl comprising five or six ring members, of which between one and four said members may be heteroatoms chosen from N, O, and S, or 2) bicyclic heteroaryl, wherein each of the fused rings comprises five or six ring members, comprising between them one to four heteroatoms chosen from N, O, and S.

The term “lower cycloalkyl,” as used herein, alone or in combination, means a monocyclic cycloalkyl having between three and six ring members (i.e., C₃-C₆ cycloalkyl). Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms chosen from N, O, and S (i.e., C₃-C₆ heterocycloalkyl). Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently chosen from hydrogen and lower alkyl, either of which may be optionally substituted.

The term “mercaptyl” as used herein, alone or in combination, refers to an RS-group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently chosen from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Where structurally feasible, two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety chosen from hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. For example, an unsymmetrical group such as —C(O)N(R)— may be attached to the parent moiety at either the carbon or the nitrogen.

Asymmetric centers exist in the compounds disclosed herein. These centers are designated by the symbol “R” or “S,” depending on the configuration of substituents around the chiral carbon atom. It should be understood that the disclosure encompasses all stereochemical isomeric forms, including diastereomeric, enantiomeric, and epimeric forms, as well as d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present disclosure includes all cis, trans, syn, anti, entgegen (E), and zusammen (Z) isomers as well as the appropriate mixtures thereof. Additionally, compounds may exist as tautomers; all tautomeric isomers are provided by this disclosure. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “IC₅₀” is that concentration of inhibitor which reduces the activity of an enzyme to half-maximal level.

The term polycomb group of ring finger protein (“PCGF”), as used herein, alone or in combination, refers to one of the two types of proteins that characterize PRC1. There are at least six variants of PCGF proteins, commonly termed PCGF1-PCGF6. In addition, the PCGF4 variant is also termed BMI-1.

The term “polycomb repressive complex 1” (PRC1) as used herein, alone or in combination, refers to a complex containing a RNF1 or RNF2 component, and a polycomb group of ring finger (PCGF) protein, which combined confer E3 ubiquitin ligase activity towards Lys119 on histone H2A. Due to the presence of multiple paralogues, human PRC1 complexes can occur in several combinations, corresponding to the six PCGF proteins and two RNF1 proteins. PRC1 contains additional subunits which define two subclasses: canonical PRC1, which contains a chromobox (“CBX”) protein, and noncanonical PRC1, which contains either the RING1B and YY1 binding protein (“RYBP”) or the YAF2 homolog.

“PRC1 inhibitor” is used herein to refer to a compound that exhibits an IC₅₀ with respect to PRC1 activity of no more than 20 μM, as measured in the PRC1 assay described generally herein. Certain compounds disclosed herein have been discovered to exhibit inhibition against PRC1. In certain embodiments, compounds will exhibit an IC₅₀ with respect to PRC1 of no more than about 10 μM; in further embodiments, compounds will exhibit an IC₅₀ with respect to PRC1 of no more than about 1 μM; in yet further embodiments, compounds will exhibit an IC₅₀ with respect to PRC1 of not more than about 200 nM; in yet further embodiments, compounds will exhibit an IC₅₀ with respect to PRC1 of not more than about 50 nM, as measured in the PRC1 assay described herein.

The term “PRC1 catalytic inhibitor” is used herein to refer to a compound that targets a RNF1 or RNF2 subunit of the PRC1 complex, and exhibits an IC₅₀ of no more than about 100 μM, as measured in the assay described generally herein. In certain embodiments, the PRC1 catalytic inhibitor exhibits an IC₅₀ of 50 μM or lower. In certain embodiments, the PRC1 catalytic inhibitor exhibits an IC₅₀ of 20 μM or lower. In certain embodiments, the PRC1 catalytic inhibitor exhibits an IC₅₀ of 10 μM or lower. In certain embodiments, the PRC1 catalytic inhibitor exhibits an IC₅₀ of 5 μM or lower. In certain embodiments, the PRC1 catalytic inhibitor exhibits an IC₅₀ of 1 μM or lower. In certain embodiments, the PRC1 catalytic inhibitor exhibits an IC₅₀ of 200 nM or lower.

The term “RING finger domain” refers to a zinc finger domain comprising Cys and/or His zinc binding residues that is often involved in the ubiquitination of proteins.

The term “RNF1” refers to the ring finger protein 1 found in PRC1. “RNF1” is alternatively termed “RING1” or “RING1A” in the literature.

The term “RNF2” refers to the ring finger protein 2 found in PRC1. “RNF2” is alternatively termed “RING2” or “RING1B” in the literature.

In certain embodiments, the compounds may exert their therapeutic efficacy by inhibiting canonical PRC1. In other embodiments, the compounds may act by inhibiting non-canonical PRC1. Inhibiting both canonical and non-canonical PRC1 as measured by the assay described above should provide the basis for maximal therapeutic efficacy.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present disclosure includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine. Other representative organic amines useful for the formation of base addition salts include ethylenediamine, ethanolamine, diethanolamine, piperidine, and piperazine.

### Pharmaceutical Compositions

While it may be possible for the compounds of the subject disclosure to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject disclosure or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

### Oral Administration

The compounds of the present disclosure may be administered orally, including swallowing, so the compound enters the gastrointestinal tract, or is absorbed into the blood stream directly from the mouth, including sublingual or buccal administration.

Suitable compositions for oral administration include solid formulations such as tablets, pills, cachets, lozenges and hard or soft capsules, which can contain liquids, gels, powders, or granules, solutions or suspensions in an aqueous liquid or a non-aqueous liquid, or as an oil-in-water liquid emulsion or a water-in-oil liquid emulsion. The active ingredient may also be presented as a bolus, electuary or paste.

In a tablet or capsule dosage form the amount of drug present may be from about 0.05% to about 95% by weight, more typically from about 2% to about 50% by weight of the dosage form.

In addition, tablets or capsules may contain a disintegrant, comprising from about 0.5% to about 35% by weight, more typically from about 2% to about 25% of the dosage form. Examples of disintegrants include methyl cellulose, sodium or calcium carboxymethyl cellulose, croscarmellose sodium, polyvinylpyrrolidone, hydroxypropyl cellulose, starch and the like.

Suitable binders, for use in a tablet, include gelatin, polyethylene glycol, sugars, gums, starch, hydroxypropyl cellulose and the like. Suitable diluents, for use in a tablet, include mannitol, xylitol, lactose, dextrose, sucrose, sorbitol and starch.

Suitable surface active agents and glidants, for use in a tablet or capsule, may be present in amounts from about 0.1% to about 3% by weight, and include polysorbate 80, sodium dodecyl sulfate, talc and silicon dioxide.

Suitable lubricants, for use in a tablet or capsule, may be present in amounts from about 0.1% to about 5% by weight, and include calcium, zinc or magnesium stearate, sodium stearyl fumarate and the like.

Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with a liquid diluent. Dyes or pigments may be added to tablets for identification or to characterize different combinations of active compound doses.

Liquid formulations can include emulsions, solutions, syrups, elixirs and suspensions, which can be used in soft or hard capsules. Such formulations may include a pharmaceutically acceptable carrier, for example, water, ethanol, polyethylene glycol, cellulose, or an oil. The formulation may also include one or more emulsifying agents and/or suspending agents.

Compositions for oral administration may be formulated as immediate or modified release, including delayed or sustained release, optionally with enteric coating.

In another embodiment, a pharmaceutical composition comprises a therapeutically effective amount of a compound of Formula (I) or a pharmaceutically acceptable salt thereof, and a pharmaceutically acceptable carrier.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

### Parenteral Administration

Compounds of the present disclosure may be administered directly into the blood stream, muscle, or internal organs by injection, e.g., by bolus injection or continuous infusion. Suitable means for parenteral administration include intravenous, intra-muscular, subcutaneous intraarterial, intraperitoneal, intrathecal, intracranial, and the like. Suitable devices for parenteral administration include injectors (including needle and needle-free injectors) and infusion methods. The formulations may be presented in unit-dose or multi-dose containers, for example sealed ampoules and vials.

Most parenteral formulations are aqueous solutions containing excipients, including salts, buffering, suspending, stabilizing and/or dispersing agents, antioxidants, bacteriostats, preservatives, and solutes which render the formulation isotonic with the blood of the intended recipient, and carbohydrates.

Parenteral formulations may also be prepared in a dehydrated form (e.g., by lyophilization) or as sterile non-aqueous solutions. These formulations can be used with a suitable vehicle, such as sterile water. Solubility-enhancing agents may also be used in preparation of parenteral solutions. Compositions for parenteral administration may be formulated as immediate or modified release, including delayed or sustained release. Compounds may also be formulated as depot preparations. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

### Topical Administration

Compounds of the present disclosure may be administered topically (for example to the skin, mucous membranes, ear, nose, or eye) or transdermally. Formulations for topical administration can include, but are not limited to, lotions, solutions, creams, gels, hydrogels, ointments, foams, implants, patches and the like. Carriers that are pharmaceutically acceptable for topical administration formulations can include water, alcohol, mineral oil, glycerin, polyethylene glycol and the like. Topical administration can also be performed by, for example, electroporation, iontophoresis, phonophoresis and the like.

Typically, the active ingredient for topical administration may comprise from 0.001% to 10% w/w (by weight) of the formulation. In certain embodiments, the active ingredient may comprise as much as 10% w/w; less than 5% w/w; from 2% w/w to 5% w/w; or from 0.1% to 1% w/w of the formulation.

Compositions for topical administration may be formulated as immediate or modified release, including delayed or sustained release.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

### Rectal, Buccal, and Sublingual Administration

Suppositories for rectal administration of the compounds of the present disclosure can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

### Administration by Inhalation

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the disclosure may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Other carrier materials and modes of administration known in the pharmaceutical art may also be used. Pharmaceutical compositions of the disclosure may be prepared by any of the well-known techniques of pharmacy, such as effective formulation and administration procedures. Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. In addition, the route of administration may vary depending on the condition and its severity. The above considerations concerning effective formulations and administration procedures are well known in the art and are described in standard textbooks.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

### Combinations and Combination Therapy

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

In another aspect, a compound with PRC1 inhibitory properties, as disclosed herein, is optionally used in combination with procedures that provide additional benefit to the patient. The inhibitor and any additional therapies are optionally administered before, during, or after the occurrence of a disease or condition, and the timing of administering the composition containing the inhibitor varies in some embodiments. Thus, for example, the inhibitor may be used as a prophylactic and is administered continuously to subjects with a propensity to develop conditions or diseases in order to prevent the occurrence of the disease or condition. The inhibitor and compositions are optionally administered to a subject during or as soon as possible after the onset of the symptoms.

Considering that a compound with PRC1 inhibitory properties is anticipated to target the cancer stem cells within a malignancy, it may be optimally used in combination with therapies that target instead the remaining bulk tumor cells. Therefore, for use in the treatment or attenuation of cancer and neoplastic diseases, a compound with PRC1 inhibitory properties, as disclosed herein, may be optimally used together with one or more of the
following non-limiting examples of anti-cancer agents, including, but not limited to: 1) inhibitors or modulators of a protein involved in one or more of the DNA damage repair (DDR) pathways such as:
   a. PARP1/2, including, but not limited to: olaparib, niraparib, rucaparib;
   b. checkpoint kinase 1 (CHK1), including, but not limited to: UCN-01, AZD7762, PF477736, SCH900776, MK-8776, LY2603618, V158411, and EXEL-9844;
   c. checkpoint kinase 2 (CHK2), including, but not limited to: PV1019, NSC 109555, and VRX0466617;
   d. dual CHK1/CHK2, including, but not limited to: XL-844, AZD7762, and PF-473336;
   e. WEE1, including, but not limited to: MK-1775 and PD0166285;
   f. ATM, including, but not limited to KU-55933,
   g. DNA-dependent protein kinase, including, but not limited to NU7441 and M3814; and
   h. Additional proteins involved in DDR;
2) Inhibitors or modulators of one or more immune checkpoints, including, but not limited to:
   a. PD-1 inhibitors such as nivolumab (OPDIVO), pembrolizumab (KEYTRUDA), pidilizumab (CT-011), and AMP-224 (AMPLIMMUNE);
   b. PD-L1 inhibitors such as Atezolizumab (TECENTRIQ), Avelumab (Bavencio), Durvalumab (Imfinzi), MPDL3280A (Tecentriq), BMS-936559, and MEDI4736;
   c. anti-CTLA-4 antibodies such as ipilimumab (YERVOY) and CP-675,206 (TREMELIMUMAB);
   d. inhibitors of T-cell immunoglobulin and mucin domain 3 (Tim-3);
   e. inhibitors of V-domain Ig suppressor of T cell activation (Vista);
   f. inhibitors of band T lymphocyte attenuator (BTLA);
   g. inhibitors of lymphocyte activation gene 3 (LAG3); and
   h. inhibitors of T cell immunoglobulin and immunoreceptor tyrosine-based inhibitory motif domain (TIGIT);
3) telomerase inhibitors or telomeric DNA binding compounds;
4) alkylating agents, including, but not limited to: chlorambucil (LEUKERAN), oxaliplatin (ELOXATIN), streptozocin (ZANOSAR), dacarbazine, ifosfamide, lomustine (CCNU), procarbazine (MATULAN), temozolomide (TEMODAR), and thiotepa;
5) DNA crosslinking agents, including, but not limited to: carmustine, chlorambucil (LEUKERAN), carboplatin (PARAPLATIN), cisplatin (PLATIN), busulfan (MYLERAN), melphalan (ALKERAN), mitomycin (MITOSOL), and cyclophosphamide (ENDOXAN);
6) anti-metabolites, including, but not limited to: cladribine (LEUSTATIN), cytarbine, (ARA-C), mercaptopurine (PURINETHOL), thioguanine, pentostatin (NIPENT), cytosine arabinoside (cytarabine, ARA-C), gemcitabine (GEMZAR), fluorouracil (5-FU, CARAC), capecitabine (XELODA), leucovorin (FUSILEV), methotrexate (RHEUMATREX), and raltitrexed;
7) antimitotics, which are often plant alkaloids and terpenoids, or derivateves thereof including but limited to: taxanes such as docetaxel (TAXITERE), paclitaxel (ABRAXANE, TAXOL), vinca alkaloids such as vincristine (ONCOVIN), vinblastine, vindesine, and vinorelbine (NAVELBINE);
8) topoisomerase inhibitors, including, but not limited to: amsacrine, camptothecin (CTP), genisten, irinotecan (CAMPTOSAR), topotecan (HYCAMTIN), doxorubicin (ADRIAMYCIN), daunorubicin (CERUBIDINE), epirubicin (ELLENCE), ICRF-193, teniposide (VUMON), mitoxantrone (NOVANTRONE), and etoposide (EPOSIN);
9) DNA replication inhibitors, including, but not limited to: fludarabine (FLUDARA), aphidicolin, ganciclovir, and cidofovir;
10) ribonucleoside diphosphate reductase inhibitors, including, but not limited to: hydroxyurea;
11) transcription inhibitors, including, but not limited to: actinomycin D (dactinomycin, COSMEGEN) and plicamycin (mithramycin);
12) DNA cleaving agents, including, but not limited to: bleomycin (BLENOXANE), idarubicin,
13) cytotoxic antibiotics, including, but not limited to: actinomycin D (dactinomycin, COSMEGEN),
14) aromatase inhibitors, including, but not limited to: aminoglutethimide, anastrozole (ARIMIDEX), letrozole (FEMARA), vorozole (RIVIZOR), and exemestane (AROMASIN);
15) angiogenesis inhibitors, including, but not limited to: genistein, sunitinib (SUTENT), and bevacizumab (AVASTIN);
16) anti-steroids and anti-androgens, including, but not limited to: aminoglutethimide (CYTADREN), bicalutamide (CASODEX), cyproterone, flutamide (EULEXIN), nilutamide (NILANDRON);
17) tyrosine kinase inhibitors, including, but not limited to: imatinib (GLEEVEC), erlotinib (TARCEVA), lapatininb (TYKERB), sorafenib (NEXAVAR), and axitinib (INLYTA);
18) mTOR inhibitors, including, but not limited to: everolimus, temsirolimus (TORISEL), and sirolimus;
19) monoclonal antibodies, including, but not limited to: trastuzumab (HERCEPTIN) and rituximab (RITUXAN);
20) apoptosis inducers such as cordycepin;
21) protein synthesis inhibitors, including, but not limited to: clindamycin, chloramphenicol, streptomycin, anisomycin, and cycloheximide;
22) antidiabetics, including, but not limited to: metformin and phenformin;
23) antibiotics, including, but not limited to:
   a. tetracyclines, including, but not limited to: doxycycline;
   b. erythromycins, including, but not limited to: azithromycin;
   c. glycylglycines, including, but not limited to: tigecyline;
   d. antiparasitics, including, but not limted to: pyrvinium pamoate;
   e. beta-lactams, including, but not limited to the penicillins and cephalosporins;
   f. anthracycline antibiotics, including, but not limited to: daunorubicin and doxorubicin;
   g. other antibiotics, including, but not limited to: chloramphenicol, mitomycin C, and actinomycin;
24) antibody therapeutical agents, including, but not limited to: muromonab-CD3, infliximab (REMICADE), adalimumab (HUMIRA), omalizumab (XOLAIR), daclizumab (ZENAPAX), rituximab (RITUXAN), ibritumomab (ZEVALIN), tositumomab (BEXXAR), cetuximab (ERBITUX), trastuzumab (HERCEPTIN), ADCETRIS, alemtuzumab (CAMPATH-1H), Lym-1 (ONCOLYM), ipilimumab (YERVOY), vitaxin, bevacizumab (AVASTIN), and abciximab (REOPRO); and
25) other agents, such as Bacillus Calmette-Gudrin (B-C-G) vaccine; buserelin (ETILAMIDE); chloroquine (ARALEN); clodronate, pamidronate, and other bisphosphonates; colchicine; demethoxyviridin; dichloroacetate; estramustine; filgrastim (NEUPOGEN); fludrocortisone (FLORINEF); goserelin (ZOLADEX); interferon; leucovorin; leuprolide (LUPRON); levamisole; lonidamine; mesna; metformin; mitotane (o,p′-DDD, LYSODREN); nocodazole; octreotide (SANDOSTATIN); perifosine; porfimer (particularly in combination with photo- and radiotherapy); suramin; tamoxifen; titanocene dichloride; tretinoin; anabolic steroids such as fluoxymesterone (HALOTESTIN); estrogens such as estradiol, diethylstilbestrol (DES), and dienestrol; progestins such as medroxyprogesterone acetate (MPA) and megestrol; and testosterone;

Where a subject is suffering from or at risk of suffering from an inflammatory condition, a compound with PRC1 inhibitory properties, as disclosed herein, is optionally used together with one or more agents or methods for treating an inflammatory condition in any combination. Therapeutic agents/treatments for treating an autoimmune and/or inflammatory condition include, but are not limited to any of the following examples:
1) corticosteroids, including but not limited to cortisone, dexamethasone, and methylprednisolone;
2) nonsteroidal anti-inflammatory drugs (NSAIDs), including but not limited to ibuprofen, naproxen, acetaminophen, aspirin, fenoprofen (NALFON), flurbiprofen (ANSAID), ketoprofen, oxaprozin (DAYPRO), diclofenac sodium (VOLTAREN), diclofenac potassium (CATAFLAM), etodolac (LODINE), indomethacin (INDOCIN), ketorolac (TORADOL), sulindac (CLINORIL), tolmetin (TOLECTIN), meclofenamate (MECLOMEN), mefenamic acid (PONSTEL), nabumetone (RELAFEN) and piroxicam (FELDENE);
3) immunosuppressants, including but not limited to methotrexate (RHEUMATREX), leflunomide (ARAVA), azathioprine (IMURAN), cyclosporine (NEORAL, SANDIMMUNE), tacrolimus and cyclophosphamide (CYTOXAN);
4) CD20 blockers, including but not limited to rituximab (RITUXAN);
5) Tumor Necrosis Factor (TNF) blockers, including but not limited to etanercept (ENBREL), infliximab (REMICADE) and adalimumab (HUMIRA);
6) interleukin-1 receptor antagonists, including but not limited to anakinra (KINERET);
7) interleukin-6 inhibitors, including but not limited to tocilizumab (ACTEMRA);
8) interleukin-17 inhibitors, including but not limited to AIN457;
9) Janus kinase inhibitors, including but not limited to tasocitinib; and
10) syk inhibitors, including but not limited to fostamatinib.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

### Indications

Thus, in another aspect, certain embodiments provide methods for treating PRC1-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of PRC1-mediated disorders.

The compounds, compositions, and methods disclosed herein are useful for the treatment of disease. In certain embodiments, the disease is one of dysregulated cellular proliferation, including cancer. The cancer may be hormone-dependent or hormone-resistant, such as in the case of breast cancers. In certain embodiments, the cancer is a solid tumor. In other embodiments, the cancer is a lymphoma or leukemia. In certain embodiments, the cancer is and a drug resistant phenotype of a cancer disclosed herein or known in the art. Tumor invasion, tumor growth, tumor metastasis, and angiogenesis may also be treated using the compositions and methods disclosed herein. Precancerous neoplasias are also treated using the compositions and methods disclosed herein.

Cancers to be treated by the methods disclosed herein include colon cancer, breast cancer, ovarian cancer, lung cancer, and prostate cancer; cancers of the oral cavity and pharynx (lip, tongue, mouth, larynx, pharynx), esophagus, stomach, small intestine, large intestine, colon, rectum, liver and biliary passages; pancreas, bone, connective tissue, skin, cervix, uterus, corpus endometrium, testis, bladder, kidney and other urinary tissues, including renal cell carcinoma (RCC); cancers of the eye, brain, spinal cord, and other components of the central and peripheral nervous systems, as well as associated structures such as the meninges; and thyroid and other endocrine glands.

### Solid Tumors

Cancers to be treated by the methods disclosed herein include solid tumors such as cancers of the lung, bronchus, oral cavity, and pharynx, cancers of the breast, colon, kidney, bladder, and rectum, cancers of the digestive system, including cholangiocarcinoma and stomach, esophagus, liver, and intrahepatic bile duct cancers, brain and other nervous system cancers, head and neck cancers, cancers of the cervix, uterine corpus, thyroid, ovary, testes, and prostate; thymoma, and skin cancers, including basal cell carcinoma, squamous cell carcinoma, actinic keratosis, and melanoma.

### Hematologic Cancers

The term “cancer” also encompasses cancers that do not necessarily form solid tumors, including Hodgkin's disease, non-Hodgkin's lymphomas, multiple myeloma and hematopoietic malignancies including leukemias (Chronic Lymphocytic Leukemia (CLL), Acute Lymphocytic Leukemia (ALL), Chronic Myelogenous Leukemia (CML), Acute Myelogenous Leukemia (AML),) lymphomas including lymphocytic, granulocytic and monocytic, and plasma cell neoplasms, lymphoid neoplasms and cancers associated with AIDS.

Hematological cancers include leukemia and malignant lymphoproliferative conditions that affect blood, bone marrow and the lymphatic system. Leukemia can be classified as acute leukemia and chronic leukemia. Acute leukemia includes acute lymphoid leukemia (ALL) and acute myelogenous leukemia (AML). Chronic leukemia includes chronic lymphoid leukemia (CLL) and chronic myelogenous leukemia (CML). Other related conditions include myelodysplastic syndromes (MDS, formerly known as “preleukemia”) which are a diverse collection of hematological ailments united by ineffective or abnormal production of myeloid blood cells and which risk transformation to AML.

Additional types of cancers which may be treated using the compounds and methods of the invention include, but are not limited to, adenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, head and neck cancer, hemangioblastoma, hepatocellular carcinoma, hepatoma, Kaposi's sarcoma, large cell carcinoma, leiomyosarcoma, leukemias, liposarcoma, lymphatic system cancer, lymphomas, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, paraganglioma, parathyroid tumors, pheochromocytoma, pinealoma, plasmacytomas, retinoblastoma, rhabdomyosarcoma, sebaceous gland carcinoma, seminoma, skin cancers, melanoma, small cell lung carcinoma, non-small cell lung carcinoma, squamous cell carcinoma, sweat gland carcinoma, synovioma, thyroid cancer, uveal melanoma, and Wilm's tumor.

In certain embodiments, the compositions and methods disclosed herein are useful for the treatment of a cancer chosen from AML, CML, ALL, CLL, mantle cell lymphoma, squamous cell carcinoma, Kaposi's sarcoma, osteosarcoma, endometrial cancer, ovarian cancer, breast cancer (including estrogen receptor positive breast cancer), head & neck cancer (including glioma, glioblastoma, and medulloblastoma), lung cancer (including non-small cell lung cancer and lung adenocarcinoma), digestive tract cancer, biliary tract cancer, oral or tongue cancer, liver cancer (including hepatocarcinoma), colorectal cancer, bladder cancer, pancreatic cancer (including pancreatic ductal adenocarcinoma).

In certain embodiments, the compositions and methods disclosed herein are useful for the treatment of a cancer chosen from leukemia, mantle cell lymphoma, medulloblastoma, Kaposi's sarcoma, endometrial cancer, ovarian cancer, breast cancer, squamous cell carcinoma, lung adenocarcinoma, and biliary tract cancer.

In certain embodiments, the compositions and methods disclosed herein are useful for the treatment of prostate cancer, including metastatic prostate cancer, androgen receptor pathway active prostate cancer, neuroendocrine prostate cancer, and double negative prostate cancer.

In certain embodiments, the compositions and methods disclosed herein are useful for preventing or reducing tumor invasion and tumor metastasis.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### Example 1: 2-(4-aminophenethyl)isoindoline-1,3-dione (Compound 1 or (1))

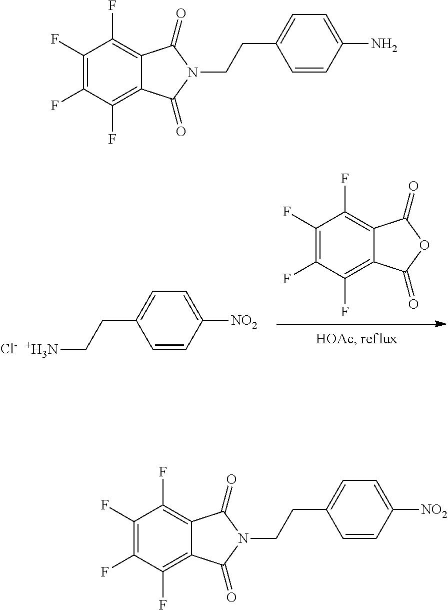 [SMILES: Nc1ccc(CCN2C(=O)c3c(F)c(F)c(F)c(F)c3C2=O)cc1 ; [Cl-] ; [NH3+]CCc1ccc([N+](=O)[O-])cc1 ; O=C1OC(=O)c2c(F)c(F)c(F)c(F)c21 ; O=C1c2c(F)c(F)c(F)c(F)c2C(=O)N1CCc1ccc([N+](=O)[O-])cc1]

2-(4-Nitrophenethyl)isoindoline-1,3-dione Tetrafluoro phthalic anhydride (1.13 g, 6.09 mmol, 1.3 equiv.) was added to a solution of 4-nitrophenethylamine hydrochloride (1 g, 4.68 mmol) in HOAc (40 ml), and the resulting mixture was refluxed overnight. After cooling to room temperature, the solvent was removed under vacuum. The residue was then dissolved in EtOAc (100 ml), washed with water, dried over Na₂SO₄ and concentrated under vacuum. The mixture was purified by flash silica gel chromatography eluting with EtOAc/Hexane gradient, 40-50%, to afford the title compound (1.39 g, yield 80%) as a yellow powder.

¹H NMR (DMSO-d₆, 600 MHz): δ 8.17 (d, J=8.5 Hz, 2H), 7.41 (d, J=8.5 Hz, 2H), 3.97 (t, J=7.3 Hz, 2H), 3.1 (t, J=7.5 Hz, 2H); ¹⁹F NMR (proton decoupled, DMSO-d₆, 600 MHz): δ −139.00 (m), −144.62 (m).

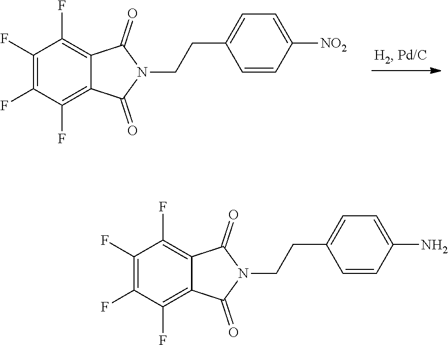 [SMILES: O=C1c2c(F)c(F)c(F)c(F)c2C(=O)N1CCc1ccc([N+](=O)[O-])cc1 ; Nc1ccc(CCN2C(=O)c3c(F)c(F)c(F)c(F)c3C2=O)cc1]

2-(4-Aminophenethyl)-4,5,6,7-tetrafluoroisoindoline-1,3-dione The product from the previous step (330 mg, 0.897 mmol) was dissolved in a 10 ml mixture of ethanol: EtOAc (1:1, v/v). The resulting solution was degassed with Ar, then quickly treated with 10% (by weight) Pd/C (90 mg, 20 wt. % loading) and the reaction was purged with H₂. Then a balloon filled with H₂ was applied to the reaction mixture through a three-way adapter under vigorous stirring. Reaction evolution was monitored by TLC. The mixture was then degassed with Ar, filtered through a thick pad of CELITE®, and washed with methanol. The solvent was removed under vacuo. The residue was purified by flash chromatography eluting with 50-70% EtOAc in hexanes to afford the title compound (250 mg, 83%) as a yellow powder.

¹H NMR (DMSO-d₆, 600 MHz): δ 6.84 (d, J=8.3 Hz, 2H), 6.46 (d, J=8.3 Hz, 2H), 4.90 (brs, 2H), 3.67 (t, J=7.2 Hz, 2H), 2.7 (t, J=7.5 Hz, 2H); ¹³C NMR (125 MHz): δ 168.03, 152.12, 134.62, 130.10, 119.64, 44.81, 38.20; ¹⁹F NMR (proton decoupled, DMSO-d₆, 600 MHz): δ −135.62 (q, J=9.6, 21.4 Hz), −142.48 (q, J=9.4, 21.6 Hz); EIMS: m/z 339.1 [M+H]⁺, calcd for C₁₆H₁₁F₄N₂O₂: 339.08.

### Example 2: 2-(pyridin-3-ylmethylene)-1H-indene-1,3(2H)-dione (Compound 2, “PRT-4165”, or (2))

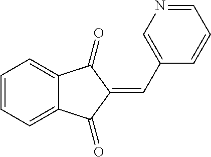 [SMILES: O=C1C(=Cc2cccnc2)C(=O)c2ccccc21]

### Example 3: N-(2,6-dibromo-4-methoxyphenyl)-4-(2-methylimidazo[1,2-a]pyrimidin-3-yl)thiazol-2-amine

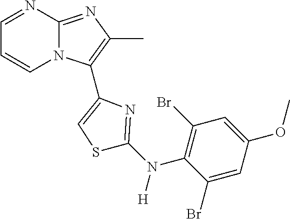 [SMILES: COc1cc(Br)c(Nc2nc(-c3c(C)nc4ncccn34)cs2)c(Br)c1]

### Example 4: Sources

### Cell Lines and Reagents

The LNCaP, 22rv1, VCaP, DU145, PC3 cells were obtained from ATCC and 293FT packaging cells from Invitrogen and cultured according to the manufacturers' instructions. PC3M cells were a gift from Dr. Raymond Bergan (formerly of Northwestern University, now OHSU Knight Cancer Institute) and cultured in RPMI-1640 supplemented with 10% Fetal Bovine Serum, 2 mM L-Glutamine (Glu), 100 IU/ml Penicillin/Streptomycin. RM1 cells were from Timothy Thompson Lab in MD Anderson Cancer Center and cultured in DMEM supplemented with 10% Fetal Bovine Serum, 2 mM L-Glutamine (Glu), 100 IU/ml Penicillin/Streptomycin. The RNF2 inhibitor PRT4165 (5047) and CCR2 antagonist RS504393 (2517) were from Tocris. The CSF-1R inhibitor BLZ945 (S7725) was from Selleckchem.

TABLE 2
Antibodies

| Reagent | Source | Identifier |
|---|---|---|
| CD44 | BD | 555478 |
| ITGB4 | MSKCC Antibody Facility | |
| RNF2 | Proteintech | 16031-1-AP |
| RNF2 | MBL | D139-3 |
| BMI1 | Cell Signaling | 6964 |
| AR | Cell Signaling | 5153 |
| AR | Santa Cruz | Sc-816 |
| E-cadherin | Cell Signaling | 3195 |
| Vimentin | Cell Signaling | 5741 |
| CD44 | Cell Signaling | 3570 |
| ITGB4 | Santa Cruz | Sc-9090 |
| GR | Cell Signaling | 3660 |
| PCGF1 | Santa Cruz | Sc-515371 |
| PHC2 | Active Motif | 39661 |
| KDM2B | Millipore | 09-864 |
| RNF1 | Cell Signaling | 13069 |
| EZH2 | Cell Signaling | 4905 |
| SUZ12 | Cell Signaling | 3737 |
| RhoGDI | Santa Cruz | Sc-360 |
| P53 | Cell Signaling | 9282 |
| P53(S15) | Cell Signaling | 9284 |
| CC3 | Cell Signaling | 9664 |
| Ki67 | BD | 550609 |
| Ki67 | Abcam | ab16667 |
| CCL2 | Invitrogen | MA5-17040 |
| H2AK119Ub | Millipore | 05-678 |
| H3K27Me3 | Millipore | 07-449 |
| H3K9Ac | Millipore | 07-352 |
| H3K27Ac | Cell Signaling | 07-360 |
| H2A | Abcam | Ab18255 |
| mcherry | Abcam | Ab167453 |
| CD45 | BioLegend | 103125 |
| CD3ε | BioLegend | 100327 |
| F4/80 | BioLegend | 123113 |
| NK1.1 | BioLegend | 108715 |
| CD11b | BioLegend | 101239 |
| CD11b | Abcam | ab133357 |
| Ly6G | BioLegend | 127607 |
| Ly6C | BioLegend | 128035 |
| Gr-1 | BioLegend | 108443 |
| Anti-goat IgG | Vector labs | BA-950 |
| Anti-mouse CTLA-4 | Bio X Cell | BE0164 |
| Anti-mouse PD-1 | Bio X Cell | BE0146 |
| Anti-rabbit IgG | Vector Labs | PK6101 |
| Anti-rat IgG | Vector Labs | PK-4004 |
| CD68 | Boster | PA1518 |
| B220 | BD | 550286 |
| CD11b | Abcam | 133357 |
| CD4 | R&D | AF554 |
| CD8 | Cell Signaling | 98941 |
| FoxP3 | eBioscience | 14-5773-82 |
| CD31 | DIA-310 | Dianova |
| NKp46 | R&D | AF2225 |
| NKp46 | R&D | AF7005 |
| iNOS | Abcam | Ab15323 |
| Arg1 | Cell Signaling | 93668 |
| Cleaved Caspase 3 | Cell Signaling | 9661 |

TABLE 3
Biological Samples

| Reagent | Source | Identifier |
|---|---|---|
| Paraffin-embedded tissue | BIOMAX.US | PR8011a |
| microarray | | PR484 |

TABLE 4
Chemicals, Peptides, and Recombinant Proteins

| Reagent | Source | Identifier |
|---|---|---|
| DAPI | Sigma Aldrich | D9542 |
| DMEM | ThermoFisher Scientific | 11965-092 |
| RPMI 1640 | ThermoFisher Scientific | 61870-036 |
| Ham's F-12K | ThermoFisher Scientific | 21127022 |
| PrEGM BulletKit | Lonza | CC-3166 |
| L-glutamine | Corning | 25005CI |
| B27 supplement | ThermoFisher Scientific | 17504044 |
| penicillin G-streptomycin | Corning | 30004CI |
| Recombinant human EGF | R&D systems | 236-EG-200 |
| Recombinant human FGF | ThermoFisher Scientific | PHG0261 |
| Accutase | Innovative Cell | AT104 |
| | Technologies | |
| Trypsin-EDTA (0.05%) | ThermoFisher Scientific | 25300054 |
| Tyramide Alexa Fluor 488 | Invitrogen | T20922 |
| Tyramide Alexa CF 594 | Biotium | 92174 |
| PRT4165 | Tocris | 5047 |
| RS504393 | Tocris | 2517 |
| BLZ945 | Selleckchem | S7725 |
| Captisol | Captisol ® | RC-0C7-020 |
| MTT | ThermoFisher Scientific | M6494 |

TABLE 5
Short Hairpins (Source: Sigma)

| Reagent | Identifier |
|---|---|
| Human RNF2 short hairpin | TRCN0000033696 |
| Human RNF2 short hairpin | TRCN0000033697 |
| Human BMI1 short hairpin | TRCN0000020155 |
| Human BMI1 short hairpin | TRCN0000020156 |
| Human CCL2 short hairpin | TRCN0000381382 |
| Human CCL2 short hairpin | TRCN0000338480 |
| Human CCR4 short hairpin | TRCN0000356811 |
| Human CCR4 short hairpin | TRCN0000356812 |
| Mouse RNF2 short hairpin | TRCN0000226018 |
| Mouse RNF2 short hairpin | TRCN0000040579 |
| Mouse BMI1 short hairpin | TRCN0000012563 |
| Mouse BMI1 short hairpin | TRCN0000012565 |
| Mouse CCL2 short hairpin | TRCN0000301702 |
| Mouse CCL2 short hairpin | TRCN00000301701 |

TABLE 6
siRNA smart pools

| Reagent | Source | Identifier |
|---|---|---|
| Human RNF2 | Dharmacon | L-006556-00-0005 |
| Human RNF1 | Dharmacon | L-006554-00-0005 |
| Human PCGF1 | Dharmacon | L-007094-00-0005 |
| Human PHC2 | Dharmacon | L-021410-00-0005 |
| Human KDM2B | Dharmacon | L-014930-00-0005 |

TABLE 7
Taqman Gene Expression Probes (Source:
ThermoFisher Scientific)

| Reagent | Identifier |
|---|---|
| RNF2 | Hs00200541_m1 |
| BMI1 | Hs00180411_m1 |
| AR | Hs00171172_m1 |
| P53 | Hs01034249_m1 |
| PHC1 | Hs01863307_s1 |
| PHC2 | Hs00189460_m1 |
| PHC3 | Hs01118132_m1 |
| PCGF1 | Hs01016642_g1 |
| PCGF2 | Hs00810639_m1 |
| PCGF3 | Hs00196998_m1 |
| PCGF5 | Hs00737074_m1 |
| PCGF6 | Hs00827882_m1 |
| Kdm2b | Hs00404800_m1 |
| RNF1 | Hs00968517_m1 |
| L3MBTL1 | Hs00210032_m1 |
| RYBP | Hs00393028_m1 |
| YAF2 | Hs00994514_m1 |
| BCOR | Hs00372378_m1 |
| CCL2 | Hs00234140_m1 |
| CYR61 | Hs00998500_g1 |
| LIF | Hs01055668_m1 |
| IL7R | Hs00233682_m1 |
| ATF3 | Hs00231069_m1 |
| LXN | Hs00220138_m1 |
| PLAU | Hs01547054_m1 |
| GDF15 | Hs00171132_m1 |
| FGFBP1 | Hs01921428_s1 |
| RELN | Hs01022646_m1 |
| NTS | Hs00175048_m1 |
| C3 | Hs00163811_m1 |
| LGR5 | Hs00969422_m1 |
| LCN2 | Hs01008571_m1 |
| CXCL1 | Hs00236937_m1 |
| GAPDH | Hs02786624_g1 |
| RNF2 | Mm00803321_m1 |
| BMI1 | Mm03053308_g1 |
| CCL2 | Mm00441242_m1 |
| CXCL1 | Mm04207460_m1 |
| ATF3 | Mm00476033_m1 |
| NTS | Mm00481140_m1 |
| LGR5 | Mm00438890_m1 |
| GAPDH | Mm99999915_g1 |

TABLE 8
Deposited Data

| Reagent | Source | Identifier |
|---|---|---|
| Raw and analyzed data | This paper | GEO: GSE103074 |

TABLE 9
Experimental Models: Cell Lines

| Reagent | Source | Identifier |
|---|---|---|
| LNCaP | ATCC | CRL-1740 |
| 22RV1 | ATCC | CRL-2505 |
| VCaP | ATCC | CRL-2876 |
| DU145 | ATCC | HTB-81 |
| PC3 | ATCC | CRL-1435 |
| PC3M | From Dr. Raymond Bergan | N/A |
| 293FT | ThermoFisher Scientific | R70007 |
| RM1 | From Timothy Thompson | N/A |

TABLE 10
Experimental Models: Organisms/Strains

| Reagent | Source | Identifier |
|---|---|---|
| BALB/c Nude mice | Charles River | 000711 |
| Nod SCID gamma mice | The Jackson Laboratory | 005557 |
| C57B6 mice | The Jackson Laboratory | |

TABLE 11
Recombinant DNA

| Reagent | Source | Identifier |
|---|---|---|
| pRK-zRNF2 | This paper | N/A |
| pRK-zmutRNF2 | This paper | N/A |

### Example 5: Mouse Tumor Models

Male BALB/c nude mice (aged 4-6 weeks) were obtained from Charles River. Male NOD SCID gamma mice (aged 4-6 weeks) were obtained from The Jackson Laboratory. All mouse studies were conducted in accordance with protocols approved by the Institutional Animal Care and Use Committee of Memorial Sloan Kettering Cancer Center (MSKCC).

For localized tumor growth assay, cells were resuspended in 100 μl PBS with Matrigel in 1:1 ratio and subcutaneously injected into both rear flanks. The volume of the s.c. xenograft was calculated as V=L×W²/2, where L and W stand for tumor length and width, respectively. For experimental metastasis assays, cells were resuspended in 100 μl 1×PBS and intracardially injected into the left ventricle with a 26 G tuberculin syringe. For bone colonization, RM1 cells were resuspended 100 μl 1×PBS and injected into the intra-femoral artery. Metastatic burden was detected through non-invasive bioluminescence imaging of experimental animals using an IVIS Spectrum.

To investigate the effect of drug treatment, compounds or antibodies were delivered twice every week or every three days through i.p. injection except BLZ945, which was delivered orally. Bioluminescence signal was measured using the ROI tool in Living Image 4.4 software (PerkinElmer).

### Example 6: Human Pathology

Paraffin-embedded tissue microarray sections with multiple cores of prostate tumors were obtained from US Biomax. Inc. The levels of expression of RNF2 and BMI1 were determined by immunohistochemical staining. RNF2 and BMI1 immunoreactivity was evaluated and scored. The expression score was determined by combining staining intensity and the percentage of immunoreactive cells.

### Example 7: Methods

### MTT Assay

Control and RNF2-silenced PC3 cells were plated at 1×10³ per well in 96 well plates for 24 hours. After 24 hours, cells were incubated in 0.5 mg/ml MTT (Invitrogen) for 2 h at 37° C. MTT crystals were dissolved in DMSO and absorbance was measured in a plate reader at 540 nm.

### Tumor Sphere Assay

Single cell suspensions of LNCaP, DU145, PC3, PC3M or RM1 cells (1,000 cells/ml) were plated on ultra-low attachment plates and cultured in serum-free PrEGM (Lonza) supplemented with 1:50 B27, 20 ng/ml bFGF and 40 ng/ml EGF for 10 days. Tumor spheres were visualized under phase contrast microscope, photographed, and counted. For serial passage, tumor spheres were collected using 70-μm cell strainers and dissociated with ACCUTASE® for 30 min at 37° C. to obtain single-cell suspensions.

### Cell Invasion Assay

Cell invasion was assayed using MATRIGEL®-coated BioCoat cell culture inserts.

### MATRIGEL® 3D Culture

Dissociated cells were incubated in PrEGM medium (Lonza) supplemented with 1:50 B27, 20 ng/ml basic fibroblast growth factor (bFGF) and 40 ng/ml EGF. A MATRIGEL® bed was prepared in a 6 well plate by putting 4 separate drops of matrigel per well (50 μl MATRIGEL® per drop). Plates were placed in 37° C. CO₂ incubator for 30 min to allow the MATRIGEL® to solidify. For each sample, 100 μl of cell suspension was mixed with 100 μl cold MATRIGEL®, and pipetted on top of the bed (50 μl each). The plates were then incubated in 37° C. for another 30 min. Warm PrEGM (2.5 ml) was then added to each well. The cells were cultured and monitored for 10-14 days with 50% medium change every 3 days. For immunostaining experiments, the cells were cultured in 8 well chamber slide. Cells were fixed with 4% paraformaldehyde for 20 min and proceed to standard immunostaining protocol.

### Biolominescent and X-ray Imaging

For bioluminescent imaging, mice were anesthetized and injected with 1.5 mg of D-luciferin retro-orbitally at the indicated times. Animals were imaged in an IVIS® 100 chamber within 5 min after D-luciferin injection, and data were recorded using LIVING IMAGE® software (Xenogen). To measure bone colonization after intracardiac injection, photon flux was calculated by using the ROI tool in the LIVING IMAGE® software. Bone metastases were further confirmed by X-Ray imaging. Mice were anesthetized with ketamine (100 mg/kg) and xylazine (10 mg/kg), placed on digital X ray Film (Scan X) and exposed at 25 kV for 15 s using a Faxitron instrument (Model MX-20; Faxitron Corp. Buffalo, Ill.).

### Immunostaining

Immunohistochemistry on paraffin-embedded sections was performed at Molecular Cytology Core Facility of Memorial Sloan Kettering Cancer Center using Discovery XT processor (Ventana Medical Systems).

The tissue sections were deparaffinized with EZPrep buffer (Ventana Medical Systems), antigen retrieval was performed with CC1 buffer (Ventana Medical Systems). Sections were blocked for 30 minutes with Background Buster solution (Innovex), followed by avidin-biotin blocking for 8 minutes (Ventana Medical Systems) (except for slides stained with CD4 and NKp46 antibodies). Sections were incubated with anti-RNF2, anti-BMI1, anti-Ki67, anti-Cleaved Caspase 3, anti-CD11b, anti-CD68, anti-CD8, anti-CD31, anti-B220, anti-FoxP3, anti-CD4, or anti-NKp46 for 5 hours, followed by 60 minutes incubation with biotinylated horse anti-rabbit IgG at 1:200 dilution (for Ki67, Cleaved Caspase 3, CD11b and CD8) or biotinylated goat anti-rat IgG at 1:200 dilution (for CD31, B220 and FoxP3) or biotinylated horse anti-goat IgG at 1:200 dilution (for CD4 and NKp46). The detection was performed with DAB detection kit (Ventana Medical Systems) according to manufacturer instruction. Slides were counterstained with hematoxylin and coverslipped with PERMOUNT™ (Fisher Scientific).

The immunofluorescent staining was performed at Molecular Cytology Core Facility of Memorial Sloan Kettering Cancer Center using Discovery XT processor (Ventana Medical Systems).

The tissue sections were deparaffinized with EZPrep buffer (Ventana Medical Systems), antigen retrieval was performed with CC1 buffer (Ventana Medical Systems). Sections were blocked for 30 minutes with Background Buster solution (Innovex), followed by avidin-biotin blocking for 8 minutes (Ventana Medical Systems).

For iNOS/CD68 or Arg1/CD68 staining, first, slides were incubated with anti-iNOS or anti-Arg1 for 5 hours, followed by 60 minutes incubation with biotinylated goat anti-rabbit IgG at 1:200 dilution. The detection was performed with Streptavidin-HRP D (part of DABMap kit, Ventana Medical Systems), followed by incubation with Tyramide Alexa Fluor 488 prepared according to manufacturer instruction with predetermined dilutions. Next, sections were incubated with anti-CD68 for 5 hours, followed by 60 minutes incubation with biotinylated goat anti-rabbit IgG at 1:200 dilution. The detection was performed with Streptavidin-HRP D (part of DABMap kit, Ventana Medical Systems), followed by incubation with Tyramide Alexa CF 594 prepared according to manufacturer instruction with predetermined dilutions. After staining slides were counterstained with DAPI for 10 min and coverslipped with MOWIOL®.

### Oncoprint and Hierarchical Clustering

Prostate cancer patient sample gene expression and amplification data were acquired from the Oncomine database and the cBioportal database. Additionally, the UCSF metastatic prostate cancer patient dataset was kindly provided by the authors (Quigley et al., Cell 2018). Z-score 2.0 was used as cut-off value to determine mRNA up/downregulation in a given sample. For the UCSF dataset, copy number alteration was called using following log 2 ratio bounds, as used in the original paper:

chr1-chr22 gain/shallow loss/deep loss: 3/1.65/0.6

chrX, chrY gain/loss: 1.4/0.6

Oncoprint was generated using sorted data of mRNA up/downregulation and gene amplification/deletion information, ordered by aberration rate (%) and classified by tumor site (primary vs. metastatic). Morpheus (available at, e.g., software.broadinstitute.org/morpheus) was used for hierarchical clustering and to heatmap generation heatmap.

### Single Sample GSEA Projections and Visualizations

Single sample GSEA was carried out using the GenePattern module ssGSEA Projection (v9) (available at, e.g., www.genepattern.org) and GraphPad Prism (v7) was used for data visualization and related statistical analysis.

### ARPC, NEPC and DNPC Classification and AR/NE Score

The principle of AR/NE/DN subtype classification proposed by Dr. Nelson's group (Bluemn et al., 2017) was followed. Briefly, androgen receptor (AR) and downstream target gene KLK3, neuroendocrine prostate cancer (NEPC) representative markers SYP and CHGA were used as determination markers. mRNA expression z-score (calculated from RPKM) was acquired from cBioportal. ARPC was defined by those whose AR and/or KLK3 mRNA z-score >0. NEPC was defined by those whose SYP and/or CHGA mRNA z-score >0. If there is overlap with ARPC and NEPC, AR score and NE score were compared and determined by the larger score. DNPC was defined by those were not ARPC nor NEPC. AR score and NE score were calculated by using the mRNA z-score of 10 AR activity genes (KLK3, KLK2, TMPRSS2, FKBP5, NKX3-1, PLPP1, PMEPA1, PART1, ALDH1A3, STEAP4) and 10 NE signature genes (SYP, CHGA, CHGB, ENO2, CHRNB2, SCG3, SCN3A, PCSK1, ELAVL4, NKX2-1).

### RNA-Seg Analysis

Data were analyzed in Partek. Total RNAs were isolated from PC3 cells. Libraries were prepared suing the standard methodology from Illumina. Generated libraries were run on a HiSeq2500 system. Raw reads were quality-checked and subsequently mapped to the human genome (hg19) using Tophat2 (2.2.4) using default settings (Langmead and Salzberg, 2012). Differential gene expression was analyzed using the DESeq2 (1.8.1) package in R using default settings (Love et al., 2014). Gene set enrichment analysis (GSEA) (Subramanian et al., 2005) was performed on a pre-ranked gene list that generated based on the gene expression changes between the RNF2 knockdown and control cells. The hallmark gene sets and GO gene sets from the Molecular Signatures Database (MSigDB v5.1) (Subramanian et al., 2005) were evaluated by GSEA with 1,000 permutations, and those significantly (FDR <0.1) enriched pathways and GO were reported using ggplot2 R package. Heatmap analysis was performed to show the gene expression patterns between the RNF2 knockdown and control repeats, using heatmap3 R package with ward2 as distance function. Gene expressions in the heatmap were transformed in logarithm scale and normalized accordingly.

### ChIP-Seg Analysis and Data Visualization

Cell nuclei from approximately 20 million formaldehyde crosslinked (1%; 10 minutes at room temperature) were isolated and chromatin was fragmented using sonicator (bioruptor). Lysate were cleared and protein-DNA complexes were isolated using target antibodies and protein-G coated magnetic beads. Chromatin IP was conducted following the standard protocol from ActiveMotif ChIP-IT High Sensitivity® (HS) Kit. Libraries were prepared according to standard Illumia protocol. Samples were sequenced at Integrated Genomics Operation Core at MSKCC.

ChIP-Seq analysis and data visualization ChIP-seq reads were trimmed by trimmomatic (v0.33; available at, e.g., www.usadellab.org/cms/?page=trimmomatic) (Bolger et al., 2014) prior to alignment, as recommended by the ChIP kit manufacturer. The trimmed reads were then aligned to the hg19 reference genome using bowtie2 (v2.3.4.2, available at, e.g., bowtie-bio.sourceforge.net/bowtie2/index.shtml) (Langmead and Salzberg, 2012). Only uniquely aligned reads were kept for downstream analysis, with duplicate reads removed by the samtools software v1.9 (Li et al., 2009). The read density matrix (+/−5 kb from the transcription start sites (TSS) of the corresponding genes) from the HOMER software (v4.10, available at, e.g., homer.ucsd.edu/homer/) (Heinz et al., 2010) was imported to the R package pheatmap for drawing heatmaps, with signal of input subtracted. Hierarchical clustering of H3K4me3 read densities and H3K27me3 read densities across the promoter regions of RNF2 active genes or the promoter regions of RNF2 repressed genes. To visualize ChIP-seq signal at individual genomic regions, the UCSC Genome Browser (available at, e.g., genome.ucsc.edu/) was used (Kent et al., 2002). Identification of significantly over-represented functional categories was done using function of “Investigate Gene Sets” from GSEA (available at, e.g., software.broadinstitute.org/gsea/msigdb/annotate.jsp) (Mootha et al., 2003).

### Immune Cell Subset Deconvolution Analysis

Intratumoral immune cell components on the SU2C mCRPC dataset was analyzed by using CIBERSORT bulk transcriptome deconvolution technique (Newman et al., 2015). CIBERSORT is a computational framework for accurately quantifying the relative levels of distinct cell types within a complex gene expression admixture. The LM22 signature gene file, consisting of 547 genes that accurately distinguish 22 mature human hematopoietic populations and activation states, including seven T cell types, naïve and memory B cells, plasma cells, NK cells, and myeloid subsets, was used. Those p<0.05 (n=86) from the total deconvolution data output (n=118) were used.

### Gene Set Enrichment Analysis

The GSEA Java program (v3.0, Subramanian et al., 2007) was used.

### Customized Library Screen

shRNA and cDNA pool was generated based on RNA-seq data from RNF2-silenced PC3 cells. shRNAs were cloned into LENG (pMSCV) vector. The number of shRNAs targeting each gene was between 3 to 6. cDNAs were cloned into pCW-neo vector. 48 hours after virus infection, PC3 cells were resuspended in 100 μl 1×PBS and intracardially injected into the left ventricle. Mice were sacrificed four weeks after injection. Tumor cells isolated from bone lesions were subjected to qRT-PCR gene expression analysis.

### Chromatin Immunoprecipitation

Chromatin IP was conducted following the standard protocol from ActiveMotif ChIP-IT High Sensitivity® (HS) Kit. Promoter enrichment was then verified through Q-PCR.

### Candidate Library Compound Screening

The candidate library was provided by the Organic Synthesis Core Facility from MSKCC. The testing concentration of candidate compounds on PC3 cells was 1 μM. RNF2 target gene expression change was used as a readout for the first round screen. Cell viability, tumor sphere formation assay and histone modification change were then used to further confirm the activity of the candidate compound.

### FACS Analysis

Control and RNF2-silenced PC3 cells were detached with ACCUTASE® and washed in blocking solution (HBSS supplemented with 10% FBS). Cell suspensions were incubated with the indicated antibodies for 45 minutes at 4° C. and analyzed by FACS.

At the end point in vivo experiment, blood and bone marrow cells were collected from each mouse and treated with Red Blood Cell lysis buffer for 5 minutes. Cells were then washed once with RPMI supplemented 2% FBS, stained with indicated antibodies and analyzed by FACS.

Analysis of Protein and mRNA Expression

For immunoblotting, cells were washed with PBS and lysed in RIPA buffer (50 mM Tris-HCl pH 7.4, 150 mM NaCl, 1 mM EDTA, 1% Triton X-100, 1% sodium deoxycholate, and 0.1% SDS) supplemented with protease inhibitors (Calbiochem) and phosphatase inhibitors (PhosSTOP, Roche Life Science). Protein concentrations were measured by using the DC Protein Assay. Total RNA was extracted using the RNeasy Mini kit coupled with RNase-free DNase set (Qiagen) and reverse transcribed with SuperScript III First-Strand Synthesis SuperMix (Invitrogen). cDNA corresponding to approximately 10 ng of starting RNA was used for one reaction. Q-PCR was performed with Taqman Gene Expression Assay (Applied Biosystems). All quantifications were normalized to endogenous GAPDH.

### Acid Extraction of Histones

PC3 cells were exposed to drugs for the indicated hours, then harvested using 0.53 mm EDTA in PBS, and washed once with cold PBS. Nuclear extracts were prepared and histones were extracted using 0.4N sulfuric acid. H2A or ubH2A was measured using the indicated antibodies.

### In Vitro Ubiquitination Assay

RNF2-PRC1 complex was immunoprecipitated from one 15 cm plate of PC3 cells. After extensive washing, the complex was pre-incubated with drugs at 4° C. for 30 minutes. UBCH5c, E1, were from Boston Biochem. Reactions were performed in 30 μl of ubiquitilation buffer (50 mM Tris, pH 7.5, 2.5 mM MgCl₂, 0.5 mM DTT) containing ubiquitin-activating enzyme 100 ng E1, 200 ng UBCH5c, 10 μg ubiquitin, 0.2 mM ATP, 1 μg H₂A, and the indicated PNF2-PRC1 complex. After incubated at 37° C. for 60 min, the reactions were then stopped by the addition of Laemmli sample buffer, and proteins were resolved by SDS-PAGE and immunoblotted using H2A antibody.

### Quantification and Statistical Analysis

Statistical analyses used R and GraphPad Prism 7 software, with a minimum of three biologically independent samples for significance. For animal experiments with subcutaneous injections, each subcutaneous tumor was an independent sample. For intracardiac injection and survival analysis, each mouse was counted as a biologically independent sample. Results are reported as mean±SD or mean±SEM. Comparisons between two groups were performed using an unpaired two-sided Student's t test (p<0.05 was considered significant). All experiments were reproduced at least three times, unless otherwise indicated.

### Example 8: PRC1 Status in Primary and Metastatic Prostate Cancer

To examine the potential role of PRC1 in prostate cancer metastasis, patient datasets comprising non-castrate and castrate primary and metastatic samples were examined. PRC1 complexes are defined by a core heterodimeric subcomplex, RING-PCGF, which induces monoubiquitination of H2A. cPRC1, which comprises CBX, HPH and RING-PCGF, is targeted to chromatin through CBX, which recognizes the H3K27me3 mark created by PRC2, and promotes chromatin condensation through HPH. In contrast, ncPRC1 complexes are targeted to chromatin through an interaction mediated by specific constituent subunits, including RYBP, BCOR, KDM2, E2F6 and L3MBTL (data not shown). It was found that several canonical and non-canonical components are selectively amplified or overexpressed in a large fraction of metastases but not in localized tumors in the Grasso dataset (Grasso et al., 2012) (data not shown). Analysis of the SU2C/PCF and Newman datasets, which include only castration-resistant metastatic samples (Robinson et al., 2015; Quigley et al., 2018), confirmed these findings. Notably, PRC1 components are altered more frequently than PRC2 components, including EZH2, in both datasets (data not shown). Consistently, analysis of a tissue microarray (n=128) demonstrated that the levels of the core PRC1 components RNF2 and BMI1 are elevated in invasive and metastatic cancers as compared to organ-confined primary tumors with no positive locoregional lymph nodes at diagnosis (data not shown).

### Example 9: GSEA Analysis of M-CRPC

To gauge the level of activation of PRC1 across M-CRPC subtypes, gene set enrichment analysis (“GSEA”) was used. Application of the classification defined by Nelson and colleagues, which are based on discrete AR_score and NE_score genesets (Bluemn et al., 2017), indicated that the SU2C/PCF dataset consists of 64% ARPC, 12% NEPC and 23% DNPC cases. These frequencies are very similar to those observed in the 2012-2016 FHCRC necropsy series, potentially reflecting the prevalent use of second-generation AR inhibitors in recent years. Multidimensional scaling analysis of the dataset using the AR_score, the NE_score, and a set of previously defined RNF2 target genes (Rai et al., 2015) revealed that the expression of RNF2 target genes was negatively correlated with that of the AR_score or NE_score, indicating that PRC1 activity is largely confined to DNPC (data not shown). Notably, PRC1's activation correlated with EMT and sternness signatures in DNPC, consistent with the hypothesis that PRC1 activity correlates with the abundance of mesenchymal-like stem cells in this prostate cancer subtype (data not shown).

To further explore this connection, a panel of androgen-dependent and AR-independent cell lines was analyzed. GSEA showed that the AR-independent PC3 and RM1 cells co-cluster with DNPC metastases from the FHCRC dataset. In contrast and as anticipated, this method classified the LNCaP, PCA2B, and VCaP cells as ARPC and the NCI-H660 as NEPC (data not shown). Curiously, the 22RV1 cells exhibited intermediate levels of AR pathway activity and the DU145 cells an intermediate NE score, pointing to potential transition states. Immunoblotting and Q-PCR analysis revealed a striking upregulation of cPRC1 and ncPRC1 components in the PC3 and PC3M cells, which possess DNPC traits and metastatic potential, as compared to the LNCaP ARPC cells (data not shown). Further investigation indicated that the LNCaP, 22RV1 and VCaP cells retain a luminal differentiation phenotype. In contrast, the DU145, PC3 and PC3M cells, which express low or undetectable levels of the AR, have lost the luminal differentiation phenotype that the AR directs, which includes the expression of E-cadherin, and have acquired expression of vimentin, suggesting that they had shed their epithelial attributes and acquired mesenchymal traits (data not shown).

In addition, it was found that the DNPC lines exhibit elevated levels of the b4 integrin (ITGB4) and CD44, which mark normal and neoplastic prostate stem cells (Yoshioka et al., 2013), and possess a higher ability to invade and form tumor spheres in vitro (data not shown). In light of the recent finding that the b4 integrin is elevated in cancer cells that have acquired stemness features by entering into an hybrid epithelial/mesenchymal state (Bierie et al., 2017), it is hypothesized that PRC1 complexes are elevated in prostate cancer cells that have become castration-resistant and metastatic through a similar process. These observations suggest that PRC1 activity correlates with the oncogenicity of prostate cancer cells that have become double negative through an incomplete EMT and the acquisition of sternness traits.

### Example 10: PRC1 is Required for Tumor Initiation and Metastasis

To investigate the role of PRC1 in prostate cancer metastasis, the obligatory E3 ligase RNF2 or the activating subunit BMI1 in PC3 cells was inactivated. It has been found that depletion of RNF2 de-stabilizes BMI1, whereas depletion of BMI1 does not affect RNF2 (data not shown). Intracardiac injection experiments indicated that the PC3 cells efficiently colonize the bone, producing predominantly osteolytic lesions similar to those occurring in AR-negative patients (Beltran et al., 2014). Intriguingly, depletion of RNF2 severely reduced metastasis in this model (data not shown). Similar results were obtained with the PC3M cells (data not shown).

To confirm and extend these results, a genetically engineered transplantation model of DNPC metastasis was developed. Transcriptomic analysis indicated that the tumors arising in Pten^{pc-/-} mice cluster between ARPC and DNPC samples, whereas the invasive and potentially metastatic tumors from Pten^{pc-/-}Smad4^{pc-/-} mice (Ding et al., 2011) completely overlap with the latter (data not shown). In agreement with these findings and their DNPC nature, the Pten^{pc-/-}Smad4^{pc-/-} tumors exhibited higher expression of mesenchymal and stem cell transcripts as compared to Pten^{pc-/-} tumors (data not shown). Moreover, late stage tumors from Pten^{pc-/-}Smad4^{pc-/-} mice consisted of large areas of AR-negative and SYP-negative DNPC and smaller areas of residual AR+ adenocarcinoma, consistent with progression from ARPC to DNPC in this model (data not shown). Finally, GSEA and transcriptional analysis as well as immunoblotting indicated that tumor cells isolated from these mice exhibit DNPC features (data not shown).

To examine the role of PRC1 in this model, RNF2 in Pten^{pc-/-}Smad4^{pc-/-} cells was depleted. As observed in PC3 cells, silencing of RNF2 destabilized BMI1 but did not reduce the expression of mesenchymal and stem cell markers or affect cell proliferation (data not shown). Intracardiac injection of Pten^{pc-/-}Smad4^{pc-/-} cells resulted in rapid formation of bone and liver metastases in syngeneic FVB/NJ mice. Importantly, depletion of RNF2 suppressed the capacity of the cells to generate metastases in these organs (data not shown). Of note, analysis of the FHCRC and Newman datasets indicated that DNPC metastases are prevalent in bone and liver but not in other distant organs (Tables 12, 13, and 14), indicating that the Pten^{pc-/-}Smad4^{pc-/-} transplantation model mimics the organotropism of human DNPC (Bluemn et al., 2017; Quigley et al., 2018). These findings indicate that inactivation of PRC1 inhibits metastasis in a genetically engineered transplantation model of DNPC.

TABLE 12
FHCRC

| | | AR+/NE− | AR−/NE+ | AR+/NE+ | AR−/NE− |
|---|---|---|---|---|---|
| All sites | | 78 | 12 | * | * |
| Lymph node | | 84 | 10 | * | * |
| Remote | Bone | 85 | 10 | * | ** |
| | Liver | 62 | 14 | 24 | ** |

TABLE 13
SU2C

| | | AR+/NE− | AR−/NE+ | AR+/NE+ | AR−/NE− |
|---|---|---|---|---|---|
| All sites | | 59 | 13 | 7 | 21 |
| Lymph node | | 68 | 14 | 10 | 8 |
| Remote | Bone | 62 | * | * | 31 |
| | Liver | 29 | 24 | 12 | 35 |

TABLE 14
UCSF

| | | AR+/NE− | AR−/NE+ | AR+/NE+ | AR−/NE− |
|---|---|---|---|---|---|
| All sites | | 65 | 8 | * | 23 |
| Lymph node | | 70 | 8 | * | 19 |
| Remote | Bone | 71 | † | † | 17 |
| | Liver | 18 | 18 | ** | 64 |

† ~10%;
* <5%;
** not detected

To further corroborate the role of PRC1 in metastasis, the RM1 cells were tested, which are derived from v-HRas v-gag-Myc transgenic prostatic tumors and exhibit activation of signaling pathways and transcriptional programs prevalent in DNPC (Power et al., 2009; Thompson et al., 1989) (data not shown). Depletion of RNF2 almost completely inhibited multi-organ site metastatic colonization (data not shown). Moreover, RNF2 knockout suppressed RM1 bone colonization upon intra-femoral artery injection, confirming that inactivation of RNF2 can suppress bone colonization even when the tumor cells are directly targeted to the bone (data not shown). These findings indicate that PRC1 is required for metastatic initiation and outgrowth in the bone and visceral organs in multiple model systems.

Given the connection between stemness and metastasis initiation, whether PRC1 promotes metastasis by regulating sternness capacity was evaluated. Indicative of a role for PRC1 in self-renewal, depletion of RNF2 or BMI1 suppressed the ability of PC3, Pten^{pc-/-}Smad4^{pc-/-}, and RM1 cells to form tumor spheres (data not shown). To more accurately determine if PRC1 affects self-renewal in vitro, control and RNF2-silenced cells were stained with PKH-26 and subjected them to serial tumor sphere assay. Consistent with the notion that the slowly cycling, label-retaining cells possess the highest self-renewal capacity (Cicalese et al., 2009), replating of the PKHHIGH, PKHPOS, and PKHNEG subsets led to sphere formation with decreasing efficiency. Notably, knockdown of RNF2 inhibited sphere formation at each passage (data not shown). Rescue experiments with a wildtype or a Ring domain deleted-RNF2 demonstrated a requirement for RNF2 catalytic activity for tumor sphere formation (data not shown). Since silencing of either RNF2 or BMI1 did not reduce CD44 or ITGB4 expression (data not shown), it may be inferred that PRC1 is not required for the specification of cancer stem cells or the expression of these markers but it specifically promotes self-renewal. Controls indicated that depletion of RNF2 does not affect proliferation of PC3, Pten^{pc-/-}Smad4^{pc-/-}, or RM1 cells under standard culture conditions, further attesting to the specificity of its effect (data not shown). This latter result is not inconsistent with the observation that inactivation of RNF2 can inhibit LNCaP cell proliferation by stabilizing TP53 (Su et al., 2013) because the PC3 and RM1 cells are TP53 mutant and the Pten^{pc-/-}Smad4^{pc-/-} cells do not exhibit detectable p53 (data not shown). These results suggest that PRC1 promotes metastasis in the context of loss of TP53, which has been linked to metastasis in genomic studies of human prostate cancer (Turajlic and Swanton, 2016).

### Example 11: Growth of RNF2-Depleted Cells in Organoid Culture

To further investigate the role of PRC1 in prostate cancer stemness, PC3 cells placed in 3D Matrigel organoid culture were studied. Whereas control PC3 cells formed invasive outgrowths in 14 days, the RNF2-depleted cells formed abortive structures containing a large fraction of apoptotic cells (data not shown). Controls indicated that inactivation of RNF2 does not impair Matrigel invasion (n=3, p=0.124), suggesting that its primary effect is to impair survival in 3D. Finally, tumor initiation experiments were performed in immunocompromised mice. Depletion of RNF2 inhibited tumor outgrowth when limiting numbers of tumor cells were inoculated, and this effect was also linked to increased apoptosis (data not shown). Based on these results, it is concluded that PRC1 sustains multiple stem cell traits in DNPC cells.

### Example 12: PRC1 Promotes the Expression of CCL2 and Other Pro-Metastatic Genes

To examine the mechanism through which PRC1 regulates the acquisition of stemness and metastatic traits, exome and ChIP sequencing studies were conducted. Depletion of RNF2 modified the expression of about 500 genes by >1.0 log 2 fold in PC3 cells. Intriguingly, 49% were down regulated while 51% were induced, suggesting that PRC1 can either promote or repress gene expression (Table S2). To integrate genome-wide occupancy of cPRC1 and ncPRC1 with control of gene expression, ChIPseq analysis was performed for RNF2 (cPRC1 and ncPRC1), BMI1 and PHC2 (cPRC1), and KDM2B (ncPRC1.1) and integrated the results with the known occupancy data for the transcriptional repression mark H3K27me3 and the activation mark H3K4me3 (GSE57498) in PC3 cells. Hierarchical clustering of RNF2-induced and suppressed genes based on H3K27me3 and H3K4me3 promoter occupancy yielded two subsets in each class (data not shown). Amongst the top 100 induced genes, 42% were found in cluster 1 and 58% in cluster 2, and amongst the top 100 repressed genes, 33% in cluster 3 and 67% in cluster 4. Cluster 1 and 3 genes were constitutively expressed at higher levels as compared to cluster 2 and 4 (data not shown). In spite of their divergent direction of regulation by RNF2, the promoters of cluster 1 and 3 genes were characterized by a higher level of the H3K4me3 activation mark as compared to those of 2 and 4. Notably, however, cluster 1 promoters, which were induced by RNF2, exhibited a lower level of H3K27me3 and of KDM2B as compared to cluster 3, consistent with a repressive role for KDM2B (data not shown). In contrast, cluster 2 and 4 promoters were characterized by lower levels of RNF2 occupancy and both H3K27me3 and H3K4me3 as compared to 1 and 3 (data not shown).

Pathway analysis of each cluster revealed that clusters 1 and 2 (induced by PRC1) are dominated by genes involved in cell adhesion and migration and genes belonging to the Extracellular Space (ES), which includes cytokines, components of the extracellular matrix, and their regulators. In contrast, cluster 3 and 4 (repressed by PRC1) comprised genes involved in metabolic pathways and genes belonging to the ES and metabolic pathways, respectively (data not shown). Consistently, pathway analysis of the global gene expression program regulated by RNF2 indicated that a large majority of genes induced by PRC1 belong to the ES category (data not shown).

### Example 13: Upregulation and Downregulation of Genes

To validate the importance of the RNF2-dependent gene expression program in prostate cancer, patient datasets were examined using a signature comprising both upregulated and downregulated genes (data not shown). Increased expression of the upregulated geneset significantly correlated with poor disease-free survival in the TCGA and Taylor datasets (Cancer Genome Atlas Research, 2015; Taylor et al., 2010) (data not shown). In contrast, increased expression of the repressed geneset did not correlate with disease-free survival (TCGA P=0.217; Taylor P=0.25). Intriguingly, GSEA indicated that expression of RNF2-activated genes correlated positively with EMT and stemness signatures and negatively with AR or NEPC signatures in the SU2C dataset (data not shown). These results suggest that the capacity of PRC1 to positively control gene expression is associated with the acquisition of mesenchymal and stem-like traits and progression to metastasis in DNPC.

### Example 14: PRC1 Promotes the Expression of CCL2 and Other Pro-Metastatic Genes

To identify PRC1 target genes involved in metastasis, a focused genetic screen was conducted in vivo by injecting RNF2-silenced PC3 cells transduced with a pool of vectors encoding the ORFs of top 5 RNF2-activated genes and multiple shRNAs targeting the top 10 RNF2-repressed genes. Four out of 10 mice developed bone metastases in 4 weeks (data not shown). Tumor cells were isolated from the lesions and subjected to q-PCR to identify the genes more consistently up- or down-regulated. Expression levels of the CC chemokine CCL2 were upregulated by about 5 fold from all 4 metastatic samples as compared to RNF2-silenced cells (data not shown). Other mediators included CXCL1, LGR5, LCN2 and C3, which have been previously implicated in tumorigenesis and metastasis (Acharyya et al., 2012; Boire et al., 2017; de Lau et al., 2014; Jung et al., 2016). However, these genes were not as largely or reproducibly up-regulated in those lesions as CCL2. Moreover, none of the repressed genes in the custom library scored positive in the screen. These findings suggest that CCL2 rescues metastatic capacity after silencing of RNF2, identifying this cytokine as the top pro-metastatic mediator controlled by PRC1.

CCL2 and the second top ranked target, CXCL1, mediate recruitment of inflammatory monocytes and their conversion into MDSCs and TAMs, which suppress immunity and promote angiogenesis and metastasis (Noy and Pollard 2014; Quayle and Joyce 2013). Moreover, both cytokines have been linked bone colonization in prostate cancer (Loberg et al. 2007; Lu et al. 2009). qPCR analysis of a panel of prostate cancer cells revealed that CCL2 mRNA levels were increased by greater than 50 fold in the DNPC PC3 and PC3M cells as compared to the AR-dependent LNCaP cells (data not shown). The changes in CCL2 expression correlated positively with those in RNF2 expression but were larger, as anticipated from an inducer-target relationship. Silencing of RNF2 or BMI1 suppressed the expression of CCL2 in both PC3 and RM1 cells, consistent with the potential identification of CCL2 as a PRC1 target gene (data not shown). Silencing of PCGF1, PHC2 and KDM2B exerted a similar effect, suggesting a participation of the ncPRC1 complex KDM2B-PRC1 in the regulation of CCL2 (data not shown). Additional experiments indicated that depletion of RNF2, RNF1A, PHC2 or KDM2B also suppresses the expression of CXCL1. As anticipated, ATF3, one of the downregulated genes, responded in opposite fashion (data not shown). Further analysis of the relative roles of canonical and ncPRC1.1 in prostate cancer metastasis revealed that depletion of BMI1 suppresses bone colonization of PC3, whereas inactivation of KDM2B almost completely blocks this process (data not shown). Survival analysis confirmed that silencing KDM2B exhibits a more profound inhibitory effect on metastasis (data not shown). The more dramatic effect of KDM2B inactivation may at least in part result from its ability to attenuate cell growth (data not shown). These results suggest that both cPRC1 and ncPRC1.1 promote prostate cancer metastasis.

To validate if CCL2 is a direct target positively regulated by PRC1, the CCL2 promoter was subjected to ChIP-qPCR with antibodies to RNF2 and various histone marks. It was found that the chromatin surrounding the CCL2 promoter is decorated by activating modifications, including H3K9ac and H3K27ac, in control PC3 cells. RNF2 depletion removed these modifications, consistent with a role for PRC1 in induction of CCL2 expression. In contrast, the repressive marks H2AK119ub and H3K27me3 were very low on the CCL2 promoter and did not change upon knockdown of RNF2 (data not shown). Similar results were obtained with PC3M cells (data not shown). As anticipated, silencing of RNF2 caused a decrease of the H2AK119ub mark and an increase of the H3K9ac and H3K27ac marks on the promoter of the PRC1-repressed gene ATF3 (data not shown). These results indicate that PRC1 directly promotes the expression of CCL2 in prostate cancer cells.

### Example 15: Targeting PRC1-CCL2 Signaling Impairs Bone Metastasis

To dissect the mechanism through which the PRC1-CCL2 axis promotes prostate cancer metastasis, it was first verified that RNF2 inactivation induces depletion of CCL2 and a concomitant decrease of CD68+ TAMs in subcutaneous PC3 tumors (data not shown). Next, the effect of RNF2 inactivation on the immune microenvironment of bone metastases was examined. Notably, RNF2 depletion not only suppressed the recruitment of TAMs but also caused a dramatic decrease in microvessel density and a large increase in NK cells (data not shown). These findings suggest that PRC1 promotes the recruitment of TAMs to the tumor stroma, creating an immunosuppressive and proangiogenic microenvironment for metastatic outgrowth.

Having considered that the PC3 cells express the CCL2 receptor CCR4, whether CCL2 could promote their capacity for self-renewal by binding to CCR4 was investigated. Intriguingly, depletion of CCL2 or CCR4 inhibited sphere formation by a similarly large degree (data not shown) although not as effectively as silencing of RNF2 (data not shown), suggesting that PRC1 promotes self-renewal at least in part by inducing CCL2. To examine the relative roles of the autocrine and paracrine effect of CCL2, CCR4 was inactivated on PC3 cells or the CCL2/CCR2 axis in monocytes/macrophages targeted. Silencing of CCR4 compromised bone metastasis, providing evidence that the increased self-renewal capacity conferred by CCL2 signaling is necessary for successful colonization of this organ (data not shown). To block the CCL2/CCR2 axis and inhibit macrophage recruitment, the selective CCR2 antagonist RS504393 or the CSF-R1 inhibitor BLZ945, respectively, were used. Bioluminescent imaging indicated that both compounds effectively inhibit the outgrowth of macroscopic bone lesions (data not shown). These results indicate that CCL2 promotes bone colonization by inducing autocrine self-renewal and by recruiting pro-tumorigenic macrophages.

To examine the consequences of inactivation of PRC1 in immune competent mice, bone sections from C57BL/6 mice inoculated with RM1 cells were stained. Silencing of RNF2 not only drastically reduced infiltration by TAMs and suppressed neoangiogenesis but also inhibited recruitment of Tregs and B cells to bone metastases (data not shown). Whereas it is well established that Tregs mediate immunosuppression in cancer (Plitas and Rudensky, 2016), B cells have been specifically implicated in prostate cancer progression (Ammirante et al., 2013). Depletion of RNF2 also induced an increase of NK cells and CD4+ T cells but not of CD8+ T cells, suggesting that this manipulation can reverse the immunosuppressive microenvironment but is insufficient to drive infiltration of effector T cells (data not shown).

To further study the connection between PRC1 activity and DNPC, a prostate cancer specific RNF2 activity score consisting of genes robustly downregulated following RNF2 depletion was built, and the SU2C dataset categorized into ARPC, DNPC, and NEPC (data not shown). Single sample GSEA showed that the RNF2 activity geneset is enriched in DNPC but not in NEPC as compared to ARPC (data not shown). Moreover, although CCL2 is not a component of the RNF2 score defined above, it was found that its expression is significantly higher in DNPC but not in NEPC (data not shown). Finally, to analyze the immune cell subsets present in DNPC, Cibersort—a deconvolution method that infers the abundance of immune cell subsets from bulk-tissue transcriptome data (Newman et al., 2015)—was used. Interestingly, the RNF2 score positively correlated with infiltration by various classes of immunocytes, including dendritic cells and M2 macrophages (data not shown). Together, these data support the conclusion that PRC1 and CCL2 drive development of an immunosuppressive tumor microenvironment in DNPC metastases.

### Example 16: Development of a Catalytic Inhibitor of PRC1

Since PRC1 promotes the expression of multiple prometastatic genes in addition to CCL2 (data not shown), inhibition of PRC1 should exert a higher therapeutic efficacy as compared to inhibition of the CCL2-CCR4 axis. Prior studies have identified the small molecule PRT4165 (2) as an inhibitor of the E3 ligase activity of PRC1 (Alchanati et al., 2009). However, this compound inhibited PRC1 activity, as assessed by monoubiquitylation of histone H2A and growth of oncospheres only at 25 μM (FIG. Error! Bookmark not defined.). Example Compound 1 was identified as a more potent PRC1 inhibitor. Titration experiments revealed that 1 inhibits H2AUb and sphere formation in PC3 cells >7.5 fold more efficiently as compared to 2 (FIG. Error! Bookmark not defined.). Example Compound 1 inhibited tumor sphere to a similar extent in RM1 cells (data not shown). As anticipated from the selective role of PRC1 in self-renewal, 1 did not inhibit cell growth under standard culture conditions when used at concentrations up to 1 μM (data not shown). Importantly, 1 inhibited RNF2-mediated H2AUb in a dose-dependent fashion in a cell-free system (data not shown). Example 1 also compared to PTC209 (3), which has been proposed to function by targeting BMI1 translation and has demonstrated activity in mouse models (Yong et al., 2016). Of note, 1 inhibited PRC1 activity more effectively as compared to 3 (data not shown). Moreover, the inhibitory effect of 1 persisted for at least 48 hours similarly to that of 3 (data not shown). These results identify 1 as a novel small molecule inhibitor of RNF2 with an apparent IC₅₀ in cells and on target of ˜0.47 μM.

To obtain an estimate of the selectivity of 1, the gene expression changes induced by 1 or 2 treatment was compared with those observed after silencing of RNF2. Pathway analysis indicated that the two molecules modified the expression of genes associated with specific cancer-relevant pathways in a similar fashion. However, consistent with its higher potency, 1 induced changes larger than those caused by 2 and by RNF2 silencing. 1 also induced changes in pathways that appeared to be not affected by RNF2 depletion and vice versa, possibly reflecting off-target effects of the molecule or differences between genetic and pharmacological modulation (data not shown). Further attesting to the potency of 1, RT-qPCR of key PRC1 targets confirmed the ability of 1 to either downregulate or upregulate them as effectively as silencing of RNF2 (FIG. Error! Bookmark not defined.).

To examine the preclinical activity of 1 as a single agent in the metastatic setting, PC3 cells were injected in mice and delivered 1 at 25 mg/kg starting from either day 7, when micrometastases can be detected histologically, or from day 21, when bioluminescent macrometases are evident in the bones. Administration of 1 from day 7 prevented formation of bone metastases, whereas treatment starting from day 21 resulted in a significant suppression of their expansion. In fact, 1 almost completely halted their growth of macrometastases during 2 weeks of treatment (FIG. Error! Bookmark not defined.). Analysis of bone sections showed that 1 substantially decreases nuclear H2AUb levels and secretion of CCL2 in the tumor microenvironment, confirming target inhibition in vivo (FIG. Error! Bookmark not defined.). 1 also inhibited the outgrowth of bone, brain and liver metastases when administered to C57BL/6 mice injected with RM1 cells (data not shown). FACS analysis on leukocytes from peripheral blood showed a significant decrease of macrophages and increase of T cells and NK cells in treated mice, suggesting that targeting PRC1 with 1 can reverse immunosuppression systemically (Figure S5I).

Pharmacological Inhibition of PRC1 Reverses Immune Suppression and Cooperates with Immunotherapy to Suppress Metastasis

To examine the hypothesis that targeting PRC1 reverses the immunosuppressive microenvironment in M-CRPC and improves the efficacy of double checkpoint immunotherapy (DCIT), the syngeneic Pten^{pc-/-}Smad4^{pc-/-} and RM1 mouse models were employed. FVB/NJ mice were inoculated intracardially with Pten^{pc-/-}Smad4^{pc-/-} cells and dosed with 1 or DCIT (anti-CTLA4 (BE0131 from bxcell.com)+anti-PD-1), singly or in combination. 1 was used at 10 mg/kg to minimize potential toxicity and better reveal cooperation with DCIT. Bioluminescent imaging clearly indicated that the combination treatment completely suppresses multi-organ metastasis, whereas 1 or DCIT used as single agents only inhibited this process (FIG. Error! Bookmark not defined. and FIG. Error! Bookmark not defined.). Survival analysis confirmed the superiority of the combination treatment (FIG. Error! Bookmark not defined.). FACS analysis of peripheral blood and bone marrow indicated that 1 reduces the numbers of MDSCs and TAMs, DCIT increases the number of T cells, and the combination exerts additive effects, indicating that the two treatment modalities have complementary systemic effects (FIG. Error! Bookmark not defined.). Similar effects were observed in RM1-injected C57BL/6 mice (Figure S6A-6G).

On-treatment staining of bone lesions revealed that 1, alone or in combination with DCIT, reduces the numbers of TAMs and Tregs (FIG. Error! Bookmark not defined. and FIG. Error! Bookmark not defined.). Double staining for CD68 and Arg1/iNOS further showed that 1 treatment dramatically decreases the percentage of M2-like TAMs, and increases the number of M1-like macrophages present at bone metastatic sites (FIG. Error! Bookmark not defined. and FIG. Error! Bookmark not defined.). In contrast, DCIT, alone or in combination with Example Compound 1, increases the recruitment of CD4+ and CD8+ T cells, whereas combination treatment inhibits the recruitment of potentially pro-tumorigenic B cells (FIG. Error! Bookmark not defined, and FIG. Error! Bookmark not defined. and FIG. Error! Bookmark not defined.). Moreover, although a significant reduction of tumor cell proliferation was not observed in any of the three treatment groups, each treatment induced apoptosis with the combination exerting the largest effect (FIG. Error! Bookmark not defined.). Overall, the combination treatment resulted in a more profound reduction of TAMs and Tregs and inhibition of neoangiogenesis and a larger increase in CD4+ and CD8+ T cells, highlighting the complementary effects of the two treatments (FIG. Error! Bookmark not defined. and FIG. Error! Bookmark not defined. and FIG. Error! Bookmark not defined. and FIG. Error! Bookmark not defined.). It may be concluded that pharmacological inhibition of PRC1 reverses the immunosuppressive microenvironment created by myeloid cells and Tregs and cooperates with DCIT to suppress metastasis, significantly extending survival in xenograft models of AR-independent CRPC.

### Discussion

Recurring cases of amplification and overexpression of multiple genes encoding PRC1 components were found in M-CRPC but not in primary tumors. GSEA indicated that these alterations potentially function in concert with upstream stimuli, such as those impinging on IKKα (Ammirante et al., 2013), to selectively elevate PRC1's activity in DNPC. In contrast, prior studies have implicated EZH2 in the development of NEPC (Ku et al., 2017). Consistently, the SU2C dataset, which includes predominantly patients treated with enzalutamide and abiraterone, exhibits a proportion of DNPC as high as that reported for the contemporary (2012-2016) FHCRC cohort (Bluemn et al., 2017). The more recent UCSF dataset (2013-2017) comprises an even higher percentage of DNPC. Intriguingly, expression of PRC1 targets correlated with EMT and sternness traits in patient samples, and in vitro studies revealed that human prostate cancer cell lines classified as DNPC possess similar traits. Moreover, PRC1 components were particularly elevated in metastatic lines. These observations suggest that PRC1 may sustain the oncogenicity of prostate cancer cells that are refractory to 2nd generation AR inhibitors because they have shed luminal adenocarcinoma features, including robust expression of the AR, and acquired mesenchymal and stem-like transcriptional traits in support of metastatic capacity. Notably, depletion of PRC1 not only inhibited the ability of metastatic AR-independent cell lines to form tumor spheres in suspension and produce invasive outgrowths in 3D Matrigel, as it could have been inferred from prior studies (Lukacs et al., 2010), but it also suppressed metastatic colonization of the bone and visceral organs through a coordinated effect on metastasis initiation and on the recruitment of TAMs and other immunosuppressive leukocytes.

By combining genome-wide occupancy analysis with expression profiling, it was found that cPRC1 associates more robustly with the promoter of RNF2-activated genes, whereas KDM2B binds more extensively to the promoters of RNF2-repressed genes. This suggests that at least in prostate cancer cells, cPRC1 mediates activation of gene expression at a genome-wide level. In contrast, ncPRC1.1 appears to be predominantly involved in gene repression. This said, ChIP Q-PCR analysis revealed that the induction of the major pro-metastatic targets of PRC1, CCL2 and CXCL1, requires not only cPRC1 but also ncPRC1.1. In consonance with these results, inactivation of either BMI1 or KDM2B suppressed prostate cancer metastasis. Given the multitude of potentially pro-metastatic genes regulated by PRC1 and the existence of additional ncPRC1, their participation in the prometastatic program governed by PRC1 cannot be excluded.

Through a focused genetic screen and subsequent mechanistic studies, CCL2 was identified as the major target of PRC1 and showed that this cytokine functions in an autocrine fashion to promote self-renewal and in a paracrine fashion to recruit TAMs at metastatic sites. Extensive evidence implicates these cells, which descend from myeloid progenitors in the bone marrow and circulate as inflammatory monocytes, in paracrine interactions that support cancer stem cells and their ability to colonize target organs (Quail and Joyce, 2013). In particular, M2-type TAMs, which are prevalent in advanced tumors, impair the maturation of dendritic cells and the activity of effector T cells, promote cancer proliferation by secreting EGF, and induce matrix remodeling and angiogenesis through production of matrix metalloproteases (Kessenbrock et al., 2010; Mantovani et al. 2017; O'Sullivan et al., 1993). Consistently, it was found that pharmacological inhibition of the CSF1-R or CCR2 on myeloid cells blocks prostate cancer metastasis, phenocopying genetic inhibition of PRC1 in tumor cells. Subsequent studies revealed that inhibition of PRC1 reverses the immunosuppression at bone metastatic sites and suppresses angiogenesis. In addition to switching macrophage polarization from M2 to M1, inhibition of PRC1 enhanced infiltration by NK cells and blocked recruitment of Tregs, which have been shown to participate in immune suppression (Plitas and Rudensky, 2016). These findings illustrate the striking ability of PRC1 to mold an immunosuppressive microenvironment at metastatic sites overcoming the barrier imposed by secondary immunoediting.

Since PRC1 regulates multiple prometastatic genes in addition to CCL2, proof-of-principle evidence that pharmacological inhibition of PRC1 may reverse immunosuppression and inhibits angiogenesis was sought. Screening of a small molecule library yielded a novel catalytic inhibitor of RNF2 with an IC50 on target and on cells of approximately 0.5 μM. The new compound, 1, suppressed H2AUb and reversed the expression of cancer-related genes controlled by PRC1. Importantly, administration of the compound not only prevented the outgrowth of bone and visceral metastasis but also curbed the expansion of established macroscopic lesions in xenograft models of M-CRPC. In depth analysis of immune competent models revealed that, although 2 suppresses the recruitment of total TAMs, it increases the number of M1-like antigen presentation-competent macrophages present at metastatic sites, removing a block to immune response and curbing neo-angiogenesis. In addition, genetic or pharmacological inhibition of PRC1 suppressed the recruitment of immunoinhibitory Tregs, presumably as a result of reduced production of CCL2 and CCL5 (Chang et al., 2016; Tan et al., 2009), and reduced infiltration by B cells. Interestingly, both types of immunocytes have been implicated in tumor progression in prostate cancer (Ammirante et al., 2013; Flammiger et al., 2013). Strikingly, although DCIT was modestly effective when used alone, in combination with 3 it provoked a substantial recruitment of CD4+ and CD8+ T cells and induced tumor cell apoptosis and metastasis regression. These results indicate that targeting PRC1's catalytic activity inhibits stemness and reverses immunosuppression in the bone and other metastatic sites.

Developmental studies have revealed that adult stem cells in various tissues recruit a variety of immune cells, including macrophages and Tregs. Once in the niche, these immune cells regulate the self-renewal and activation of stem cells to meet the diverse demands of tissue homeostasis and wound repair (Nail et al., 2018). In one such mechanism, hair follicle stem cells secrete CCL2 to attract macrophages to the bulge niche during regeneration and, reciprocally, macrophages secrete Wnt ligands to activate the stem cells (Castellana et al. 2014; Chen et al. 2015). Although the mechanisms that regulate the interaction of normal prostate stem cells with the immune system are not yet known, it is proposed herein that metastatic stem cells may highjack PRC1's function in normal stem cells to induce immunosuppression during metastasis. More broadly, the results indicate that a master epigenetic regulator, PRC1, coordinates metastasis initiation and outgrowth with suppression of both the innate and adaptive immune system and induction of neoangiogenesis. It is envisioned that targeting PRC1 may dramatically sensitize M-CRPC and other immunologically ‘cold’ cancer types to immunotherapy. Considering the role of PRC1 in promoting sternness across solid tumors and leukemias (Chan & Morey Trends Biochem. Sci. 2019), the beneficial effects of its inhibition should be widely applicable in cancer.

### REFERENCES

Bibliographic information is provided below for references cited herein. Such references, while extant at the time of filing, are not necessarily prior art.
Acharyya, S., Oskarsson, T., Vanharanta, S., Malladi, S., Kim, J., Morris, P. G., Manova-Todorova, K., Leversha, M., Hogg, N., Seshan, V. E., et al. (2012). A CXCL1 paracrine network links cancer chemoresistance and metastasis. Cell 150, 165-178.
Agudo, J., Park, E. S., Rose, S. A., Alibo, E., Sweeney, R., Dhainaut, M., Kobayashi, K. S., Sachidanandam, R., Baccarini, A., Merad, M., and Brown, B. D. (2018). Quiescent Tissue Stem Cells Evade Immune Surveillance. Immunity 48, 271-285 e275.
Alchanati, I., Teicher, C., Cohen, G., Shemesh, V., Barr, H. M., Nakache, P., Ben-Avraham, D., Idelevich, A., Angel, I., Livnah, N., et al. (2009). The E3 ubiquitin-ligase Bmi1/RNF1A controls the proteasomal degradation of Top2alpha cleavage complex—a potentially new drug target. PloS One 4, e8104.
Ammirante, M., Kuraishy, A. I., Shalapour, S., Strasner, A., Ramirez-Sanchez, C., Zhang, W., Shabaik, A., and Karin, M. (2013). An IKKalpha-E2F1-BMI1 cascade activated by infiltrating B cells controls prostate regeneration and tumor recurrence. Genes & development 27, 1435-1440.
Ammirante, M., Luo, J. L., Grivennikov, S., Nedospasov, S., and Karin, M. (2010). B-cell-derived lymphotoxin promotes castration-resistant prostate cancer. Nature 464, 302-305.
Aparicio, A. M., Shen, L., Tapia, E. L., Lu, J. F., Chen, H. C., Zhang, J., Wu, G., Wang, X., Troncoso, P., Corn, P., et al. (2016). Combined tumor suppressor defects characterize clinically defined aggressive variant prostate cancers. Clinical cancer research an official journal of the American Association for Cancer Research 22, 1520-1530.
Attard, G., Parker, C., Eeles, R. A., Schroder, F., Tomlins, S. A., Tannock, I., Drake, C. G., and de Bono, J. S. (2016). Prostate cancer. Lancet 387, 70-82.
Beer, T. M., Armstrong, A. J., Rathkopf, D. E., Loriot, Y., Sternberg, C. N., Higano, C. S., Iversen, P., Bhattacharya, S., Carles, J., Chowdhury, S., et al. (2014). Enzalutamide in metastatic prostate cancer before chemotherapy. NEJM 371, 424-433.
Beltran, H., Beer, T. M., Carducci, M. A., de Bono, J., Gleave, M., Hussain, M.,
Kelly, W. K., Saad, F., Sternberg, C., Tagawa, S. T., and Tannock, I. F. (2011). New therapies for castration-resistant prostate cancer: efficacy and safety. European urology 60, 279-290.
Beltran, H., Tomlins, S., Aparicio, A., Arora, V., Rickman, D., Ayala, G., Huang, J., True, L., Gleave, M. E., Soule, H., et al. (2014). Aggressive variants of castration-resistant prostate cancer. Clinical cancer research: an official journal of the American Association for Cancer Research 20, 2846-2850.
Ben-Saadon, R., Zaaroor, D., Ziv, T., and Ciechanover, A. (2006). The polycomb protein RNF1B generates self atypical mixed ubiquitin chains required for its in vitro histone H2A ligase activity. Molecular cell 24, 701-711.
Bierie, B., Pierce, S. E., Kroeger, C., Stover, D. G., Pattabiraman, D. R., Thiru, P., Liu Donaher, J., Reinhardt, F., Chaffer, C. L., Keckesova, Z., and Weinberg, R. A. (2017). Integrin-beta4 identifies cancer stem cell-enriched populations of partially mesenchymal carcinoma cells. PNAS USA 114, E2337-E2346.
Blackledge, N. P., Rose, N. R., and Klose, R. J. (2015). Targeting Polycomb systems to regulate gene expression: modifications to a complex story. Nature reviews molecular cell biology 16, 643-649.
Bluemn, E. G., Coleman, I. M., Lucas, J. M., Coleman, R. T., Hernandez-Lopez, S., Tharakan, R., Bianchi-Frias, D., Dumpit, R. F., Kaipainen, A., Corella, A. N., et al. (2017). Androgen receptor pathway-independent prostate cancer is sustained through fgf signaling. Cancer Cell 32, 474-489 e476.
Boire, A., Zou, Y., Shieh, J., Macalinao, D. G., Pentsova, E., and Massague, J. (2017). Complement component 3 adapts the cerebrospinal fluid for leptomeningeal metastasis. Cell 168, 1101-1113 e1113.
Bonapace, L., Coissieux, M. M., Wyckoff, J., Mertz, K. D., Varga, Z., Junt, T., and Bentires-Alj, M. (2014). Cessation of CCL2 inhibition accelerates breast cancer metastasis by promoting angiogenesis. Nature 515, 130-133.
Bos, P. D., Plitas, G., Rudra, D., Lee, S. Y., and Rudensky, A. Y. (2013). Transient regulatory T cell ablation deters oncogene-driven breast cancer and enhances radiotherapy. J. exper. med. 210, 2435-2466.
Camacho, D. F., and Pienta, K. J. (2014). A multi-targeted approach to treating bone metastases. Cancer Metastasis Reviews 33, 545-553.
Cancer Genome Atlas Research, N. (2015). The molecular taxonomy of primary prostate cancer. Cell 163, 1011-1025.
Celia-Terrassa, T., and Kang, Y. (2018). Metastatic niche functions and therapeutic opportunities. Nature Cell Biology 20, 868-877.
Chang, A. L., Miska, J., Wainwright, D. A., Dey, M., Rivetta, C. V., Yu, D., Kanojia, D., Pituch, K. C., Qiao, J., Pytel, P., et al. (2016). CCL2 produced by the glioma microenvironment is essential for the recruitment of regulatory T cells and myeloid-derived suppressor cells. Cancer research 76, 5671-5682.

de Bono, J. S., Logothetis, C. J., Molina, A., Fizazi, K., North, S., Chu, L., Chi, K. N., Jones, R. J., Goodman, O. B., Jr., Saad, F., et al. (2011). Abiraterone and increased survival in metastatic prostate cancer. NEJM, 1995-2005.
de Lau, W., Peng, W. C., Gros, P., and Clevers, H. (2014). The R-spondin/Lgr5/Rnf43 module: regulator of Wnt signal strength. Genes & development 28, 305-316.
Flammiger, A., Weisbach, L., Huland, H., Tennstedt, P., Simon, R., Minner, S., Bokemeyer, C., Sauter, G., Schlomm, T., and Trepel, M. (2013). High tissue density of FOXP3+ T cells is associated with clinical outcome in prostate cancer. Eur J Cancer 49, 1273-1279.
Gao, J., Ward, J. F., Pettaway, C. A., Shi, L. Z., Subudhi, S. K., Vence, L. M., Zhao, H., Chen, J., Chen, H., Efstathiou, E., et al. (2017). VISTA is an inhibitory immune checkpoint that is increased after ipilimumab therapy in patients with prostate cancer. Nature Medicine 23, 551-555.
Giancotti, F. G. (2013). Mechanisms governing metastatic dormancy and reactivation. Cell 155, 750-764.
Gil, J., and O'Loghlen, A. (2014). PRC1 complex diversity: where is it taking us?Trends in cell biology 24, 632-641.
Grasso, C. S., Wu, Y. M., Robinson, D. R., Cao, X., Dhanasekaran, S. M., Khan, A. P., Quist, M. J., Jing, X., Lonigro, R. J., Brenner, J. C., et al. (2012). The mutational landscape of lethal castration-resistant prostate cancer. Nature 487, 239-243.
Ismail, I. H., McDonald, D., Strickfaden, H., Xu, Z., and Hendzel, M. J. (2013). A small molecule inhibitor of polycomb repressive complex 1 inhibits ubiquitin signaling at DNA double-strand breaks. J. Biol. Chem. 288, 26944-26954.
Joshi, N. S., Akama-Garren, E. H., Lu, Y., Lee, D. Y., Chang, G. P., Li, A., DuPage, M., Tammela, T., Kerper, N. R., Farago, A. F., et al. (2015). Regulatory T Cells in Tumor-Associated Tertiary Lymphoid Structures Suppress Anti-tumor T Cell Responses. Immunity 43, 579-590.
Jung, M., Oren, B., Mora, J., Mertens, C., Dziumbla, S., Popp, R., Weigert, A., Grossmann, N., Fleming, I., and Brune, B. (2016). Lipocalin 2 from macrophages stimulated by tumor cell-derived sphingosine 1-phosphate promotes lymphangiogenesis and tumor metastasis. Sci Signal 9, ra64.
Kessenbrock, K., Plaks, V., and Werb, Z. (2010). Matrix metalloproteinases: regulators of the tumor microenvironment. Cell 141, 52-67.
Koppens, M., and van Lohuizen, M. (2016). Context-dependent actions of Polycomb repressors in cancer. Oncogene 35, 1341-1352.
Kreso, A., van Galen, P., Pedley, N. M., Lima-Fernandes, E., Frelin, C., Davis, T., Cao, L., Baiazitov, R., Du, W., Sydorenko, N., et al. (2014). Self-renewal as a therapeutic target in human colorectal cancer. Nature Medicine 20, 29-36.
Ku, S. Y., Rosario, S., Wang, Y., Mu, P., Seshadri, M., Goodrich, Z. W., Goodrich, M. M., Labbe, D. P., Gomez, E. C., Wang, J., et al. (2017). Rb1 and Trp53 cooperate to suppress prostate cancer lineage plasticity, metastasis, and antiandrogen resistance. Science 355, 78-83.
Lambert, A. W., Pattabiraman, D. R., and Weinberg, R. A. (2017). Emerging biological principles of metastasis. Cell 168, 670-691.
Langmead, B., and Salzberg, S. L. (2012). Fast gapped-read alignment with Bowtie 2. Nature Methods 9, 357-359.
Liberzon, A., Birger, C., Thorvaldsdottir, H., Ghandi, M., Mesirov, J. P., and Tamayo, P. (2015). The Molecular Signatures Database (MSigDB) hallmark gene set collection. Cell Syst 1, 417-425.
Loberg, R. D., Ying, C., Craig, M., Yan, L., Snyder, L. A., and Pienta, K. J. (2007). CCL2 as an important mediator of prostate cancer growth in vivo through the regulation of macrophage infiltration. Neoplasia 9, 556-562.
Logothetis, C. J., Gallick, G. E., Maity, S. N., Kim, J., Aparicio, A., Efstathiou, E., and Lin, S. H. (2013). Molecular classification of prostate cancer progression: foundation for marker-driven treatment of prostate cancer. Cancer Discovery 3, 849-861.
Love, M. I., Huber, W., and Anders, S. (2014). Moderated estimation of fold change and dispersion for RNA-seq data with DESeq2. Genome biology 15, 550.
Loyher, P. L., Rochefort, J., Baudesson de Chanville, C., Hamon, P., Lescaille, G., Bertolus, C., Guillot-Delost, M., Krummel, M. F., Lemoine, F. M., Combadiere, C., and Boissonnas, A. (2016). CCR2 influences T regulatory cell migration to tumors and serves as a biomarker of cyclophosphamide sensitivity. Cancer Res. 76, 6483-6494.
Lu, X., Horner, J. W., Paul, E., Shang, X., Troncoso, P., Deng, P., Jiang, S., Chang, Q., Spring, D. J., Sharma, P., et al. (2017). Effective combinatorial immunotherapy for castration-resistant prostate cancer. Nature 543, 728-732.
Lukacs, R. U., Memarzadeh, S., Wu, H., and Witte, O. N. (2010). Bmi-1 is a crucial regulator of prostate stem cell self-renewal and malignant transformation. Cell stem cell 7, 682-693.
Malladi, S., Macalinao, D. G., Jin, X., He, L., Basnet, H., Zou, Y., de Stanchina, E., and Massague, J. (2016). Metastatic latency and immune evasion through autocrine inhibition of WNT. Cell 165, 45-60.
Mantovani, A., Marchesi, F., Malesci, A., Laghi, L., and Allavena, P. (2017). Tumour-associated macrophages as treatment targets in oncology. Nature Reviews Clinical Oncology.
Mu, P., Zhang, Z., Benelli, M., Karthaus, W. R., Hoover, E., Chen, C. C., Wongvipat, J., Ku, S. Y., Gao, D., Cao, Z., et al. (2017). SOX2 promotes lineage plasticity and antiandrogen resistance in TP53- and RB1-deficient prostate cancer. Science 355, 84-88.
Nacerddine, K., Beaudry, J. B., Ginjala, V., Westerman, B., Mattiroli, F., Song,
J. Y., van der Poel, H., Ponz, O. B., Pritchard, C., Cornelissen-Steijger, P., et al. (2012). Akt-mediated phosphorylation of Bmi1 modulates its oncogenic potential, E3 ligase activity, and DNA damage repair activity in mouse prostate cancer. J. Clinical Investigation 122, 1920-1932.
Newman, A. M., Liu, C. L., Green, M. R., Gentles, A. J., Feng, W., Xu, Y., Hoang, C. D., Diehn, M., and Alizadeh, A. A. (2015). Robust enumeration of cell subsets from tissue expression profiles. Nature Methods 12, 453-457.
Noy, R., and Pollard, J. W. (2014). Tumor-associated macrophages: from mechanisms to therapy. Immunity 41, 49-61.
O'Sullivan, C., Lewis, C. E., Harris, A. L., and McGee, J. O. (1993). Secretion of epidermal growth factor by macrophages associated with breast carcinoma. Lancet 342, 148-149.
Patrawala, L., Calhoun, T., Schneider-Broussard, R., Li, H., Bhatia, B., Tang, S., Reilly, J. G., Chandra, D., Zhou, J., Claypool, K., et al. (2006). Highly purified CD44+ prostate cancer cells from xenograft human tumors are enriched in tumorigenic and metastatic progenitor cells. Oncogene 25, 1696-1708.
Pherson, M., Misulovin, Z., Gause, M., Mihindukulasuriya, K., Swain, A., and Dorsett, D. (2017). Polycomb repressive complex 1 modifies transcription of active genes. Sci. Adv. 3, e1700944.
Pienta, K. J., Machiels, J. P., Schrijvers, D., Alekseev, B., Shkolnik, M., Crabb, S. J., Li, S., Seetharam, S., Puchalski, T. A., Takimoto, C., et al. (2013). Phase 2 study of carlumab (CNTO 888), a human monoclonal antibody against CC-chemokine ligand 2 (CCL2), in metastatic castration-resistant prostate cancer. Invest New Drugs 31, 760-768.
Plitas, G., and Rudensky, A. Y. (2016). Regulatory T Cells: Differentiation and Function. Cancer Immunol Res 4, 721-725.
Quail, D. F., and Joyce, J. A. (2013). Microenvironmental regulation of tumor progression and metastasis. Nature Medicine 19, 1423-1437.
Rai, K., Akdemir, K. C., Kwong, L. N., Fiziev, P., Wu, C. J., Keung, E. Z., Sharma, S., Samant, N. S., Williams, M., Axelrad, J. B., et al. (2015). Dual roles of RNF2 in melanoma progression. Cancer Discovery 5, 1314-1327.
Robinson, D., Van Allen, E. M., Wu, Y. M., Schultz, N., Lonigro, R. J., Mosquera, J. M., Montgomery, B., Taplin, M. E., Pritchard, C. C., Attard, G., et al. (2015). Integrative clinical genomics of advanced prostate cancer. Cell 161, 1215-1228.
Rubtsov, Y. P., Rasmussen, J. P., Chi, E. Y., Fontenot, J., Castelli, L., Ye, X., Treuting, P., Siewe, L., Roers, A., Henderson, W. R., Jr., et al. (2008). Regulatory T cell-derived interleukin-10 limits inflammation at environmental interfaces. Immunity 28, 546-558.
Ryan, C. J., Smith, M. R., de Bono, J. S., Molina, A., Logothetis, C. J., de Souza, P., Fizazi, K., Mainwaring, P., Piulats, J. M., Ng, S., et al. (2013). Abiraterone in metastatic prostate cancer without previous chemotherapy. NEJM 368, 138-148.
Scher, H. I., Fizazi, K., Saad, F., Taplin, M. E., Sternberg, C. N., Miller, K., de Wit, R., Mulders, P., Chi, K. N., Shore, N. D., et al. (2012). Increased survival with enzalutamide in prostate cancer after chemotherapy. NEJM 367, 1187-1197.
Schuettengruber, B., Bourbon, H. M., Di Croce, L., and Cavalli, G. (2017). Genome regulation by polycomb and trithorax: 70 years and counting. Cell 171, 34-57.
Su, W. J., Fang, J. S., Cheng, F., Liu, C., Zhou, F., and Zhang, J. (2013). RNF2/RNF1b negatively regulates p53 expression in selective cancer cell types to promote tumor development. PNAS USA 110, 1720-1725.
Subramanian, A., Tamayo, P., Mootha, V. K., Mukherjee, S., Ebert, B. L., Gillette, M. A., Paulovich, A., Pomeroy, S. L., Golub, T. R., Lander, E. S., and Mesirov, J. P. (2005). Gene set enrichment analysis: a knowledge-based approach for interpreting genome-wide expression profiles. PNAS USA 102, 15545-15550.
Tan, M. C., Goedegebuure, P. S., Belt, B. A., Flaherty, B., Sankpal, N., Gillanders, W. E., Eberlein, T. J., Hsieh, C. S., and Linehan, D. C. (2009). Disruption of CCR5-dependent homing of regulatory T cells inhibits tumor growth in a murine model of pancreatic cancer. J Immunol 182, 1746-1755.
Taylor, B. S., Schultz, N., Hieronymus, H., Gopalan, A., Xiao, Y., Carver, B. S., Arora, V. K., Kaushik, P., Cerami, E., Reva, B., et al. (2010). Integrative genomic profiling of human prostate cancer. Cancer Cell 18, 11-22.
Thompson, T. C., Southgate, J., Kitchener, G., and Land, H. (1989). Multistage carcinogenesis induced by ras and myc oncogenes in a reconstituted organ. Cell 56, 917-930.
Wang, H., Wang, L., Erdjument-Bromage, H., Vidal, M., Tempst, P., Jones, R. S., and Zhang, Y. (2004). Role of histone H2A ubiquitination in Polycomb silencing. Nature 431, 873-878.
Yokokawa, J., Cereda, V., Remondo, C., Gulley, J. L., Arlen, P. M., Schlom, J., and Tsang, K. Y. (2008). Enhanced functionality of CD4+CD25(high)FoxP3+ regulatory T cells in the peripheral blood of patients with prostate cancer. Clinical cancer research: an official journal of the American Association for Cancer Research 14, 1032-1040.
Yong, K. J., Basseres, D. S., Welner, R. S., Zhang, W. C., Yang, H., Yan, B., Alberich-Jorda, M., Zhang, J., de Figueiredo-Pontes, L. L., Battelli, C., et al. (2016). Targeted BMI1 inhibition impairs tumor growth in lung adenocarcinomas with low CEBPalpha expression. Science translational medicine 8, 350ra104.
Yoshioka, T., Otero, J., Chen, Y., Kim, Y. M., Koutcher, J. A., Satagopan, J., Reuter, V., Carver, B., de Stanchina, E., Enomoto, K., et al. (2013). beta4 Integrin signaling induces expansion of prostate tumor progenitors. J. Clin. Investigation 123, 682-699.
Zlotnik, A., and Yoshie, O. (2012). The chemokine superfamily revisited. Immunity 36, 705-716.
Zou, M., Toivanen, R., Mitrofanova, A., Floch, N., Hayati, S., Sun, Y., Le Magnen, C., Chester, D., Mostaghel, E. A., Califano, A., et al. (2017). Transdifferentiation as a mechanism of treatment resistance in a mouse model of castration-resistant prostate cancer. Cancer Discovery 7, 736-749.

What is claimed is:

1. A method of treatment of cancer in a subject comprising the administration of an inhibitor of a RNF1 or RNF2 subunit of the polycomb repressive complex 1 (PRC1).

2. A method of prevention, reversal, or suppression of immune evasion by prostate cancer cells in a subject with cancer comprising the administration of an inhibitor of a RNF1 or RNF2 subunit of the polycomb repressive complex 1 (PRC1).

3. A method of prevention, reversal, or suppression or reversal of metastasis of cancer cells in a subject with cancer comprising the administration of an inhibitor of a RNF1 or RNF2 subunit of the polycomb repressive complex 1 (PRC1).

4. A method of prevention, reversal, or suppression or reversal of angiogenesis cells in a subject with cancer comprising the administration of an inhibitor of a RNF1 or RNF2 subunit of the polycomb repressive complex 1 (PRC1).

5. The method as recited in any one of claims 1-4, wherein the inhibitor of a RNF1 or RNF2 subunit inhibits RNF1 with an IC₅₀ of <20 μM.

6. The method as recited in claim 5, wherein the inhibitor of a RNF1 or RNF2 subunit inhibits RNF1 with an IC₅₀ of <10 μM.

7. The method as recited in claim 6, wherein the inhibitor of a RNF1 or RNF2 subunit inhibits RNF1 with an IC₅₀ of <5 μM.

8. The method as recited in claim 7, wherein the inhibitor of a RNF1 or RNF2 subunit inhibits RNF1 with an IC₅₀ of <1 μM.

9. The method as recited in any one of claims 1-4, wherein the inhibitor of a RNF1 or RNF2 subunit inhibits RNF1 with an IC₅₀ of <20 μM.

10. The method as recited in claim 9, wherein the inhibitor of a RNF1 or RNF2 subunit inhibits RNF2 with an IC₅₀ of <10 μM.

11. The method as recited in claim 10, wherein the inhibitor of a RNF1 or RNF2 subunit inhibits RNF2 with an IC₅₀ of <5 μM.

12. The method as recited in claim 11, wherein the inhibitor of a RNF1 or RNF2 subunit inhibits RNF2 with an IC₅₀ of <1 μM.

13. The method as recited in any one of claims 1-12, wherein the cancer is chosen from leukemia, mantle cell lymphoma, medulloblastoma, Kaposi's sarcoma, endometrial cancer, ovarian cancer, breast cancer, squamous cell carcinoma, lung adenocarcinoma, and biliary tract cancer.

14. The method as recited in any one of claims 1-12, wherein the cancer is prostate cancer (PC).

15. The method as recited in claim 14, wherein the prostate cancer is castration-resistant prostate cancer (CPRC).

16. The method as recited in claim 15, wherein the CPRC is androgen receptor pathway active prostate cancer.

17. The method as recited in claim 15, wherein the CPRC is neuroendocrine prostate cancer.

18. The method as recited in claim 15, wherein the CPRC is double negative prostate cancer (DNPC; AR-null NE-null prostate cancer).

19. The method of any one of claims 1-18, wherein the cancer is metastatic.

20. The method of any one of claims 1-19, additionally comprising the administration of a checkpoint inhibitor.

21. The method of claim 20, wherein the checkpoint inhibitor is a PD-1 inhibitor, PD-L1 inhibitor, or CTLA-4 inhibitor.

22. The method of claim 20, wherein the checkpoint inhibitor is chosen from nivolumab, pemborlizimab, and ipiliumumab.

23. The method of any one of claims 1-22, wherein the inhibitor of polycomb repressive complex 1 (PRC1) is a compound of structural Formula I 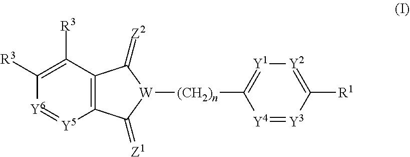
or a salt or tautomer thereof, wherein:
n is chosen from 2, 3, and 4;
W is chosen from CH and N;
Y¹, Y², Y3 and Y⁴ are independently chosen from C(R²) and N;
Y⁵, and Y⁶ are independently chosen from C(R³) and N;
Z¹ and Z² are independently chosen from ═O, ═S, —H/—OH, and —H/—H;
R¹ is chosen from amino, hydroxy, cyano, halo, alkyl, cycloalkyl, heterocycloalkyl, aryl, heteroaryl, alkoxy, cycloalkoxy, heterocycloalkoxy, aryloxy, and heteroaryloxy, any of which is optionally substituted with one or more R⁴ groups;
each R² is independently chosen from H, halo, amino, cyano, and hydroxy;
each R³ is independently chosen from H, halo, amino, cyano, and hydroxy; and
each R⁴ is independently chosen from alkyl, alkoxy, alkoxyalkyl, alkylcarbonyl, alkylsulfonyl, amino, aminocarbonyl, cyano, carboxy, halo, haloalkoxy, haloalkyl, hydroxy, hydroxyalkyl, and oxo.

24. The method as recited in claim 23, wherein Z¹ and Z² are ═O.

25. The method as recited in claim 24, wherein W is N.

26. The method as recited in claim 25, wherein each R² is independently chosen from H and halo.

27. The method as recited in claim 26, wherein Y⁵ and Y⁶ are C(R³).

28. The method as recited in claim 27, wherein at least two of R³ are halo.

29. The method as recited in claim 28, wherein R³ is chosen from H and fluoro.

30. The method as recited in claim 29, wherein R³ is fluoro.

31. The method as recited in claim 30, wherein R¹ is amino.

32. The method as recited in claim 31, wherein Y¹, Y², Y³, and Y⁴ are C(R²).
